(19) United States
(12) Patent Application Publication
(10) Pub. No.: US 2022/0152079 A1
(43) Pub. Date: May 19, 2022

(54) Methods For The Treatment Of APOC3-Related Diseases And Disorders

(71) Applicant: Arrowhead Pharmaceuticals, Inc., Pasadena, CA (US)

(72) Inventors: Bruce D. Given, Charleston, SC (US); James C. Hamilton, Sierra Madre, CA (US); Stacey Melquist, Pasadena, CA (US); Zhen Li, San Diego, CA (US); Rui Zhu, San Diego, CA (US); Tao Pei, Middleton, WI (US); Steven Kanner, Berkeley, CA (US); So Wong, Oregon, WI (US)

(21) Appl. No.: 17/592,182

(22) Filed: Feb. 3, 2022

Related U.S. Application Data
(63) Continuation of application No. PCT/US20/44879, filed on Aug. 4, 2020.
(60) Provisional application No. 62/970,613, filed on Feb. 5, 2020.
(60) Provisional application No. 62/936,559, filed on Nov. 17, 2019.
(60) Provisional application No. 62/883,046, filed on Aug. 5, 2019.

(51) Int. Cl. A61K 31/712; A61K 9/00; A61K 47/02

(52) U.S. Cl. CPC A61K 31/712; A61K 47/02; A61K 9/0019

(57) ABSTRACT

Described are methods for treating diseases and disorders that can be mediated in part by a reduction in APOC3 gene expression in a human subject in need of treatment, using pharmaceutical compositions that include APOC3 RNAi agents. The disclosed pharmaceutical compositions that include APOC3 RNAi agents, when administered to a human subject in need thereof according to the methods disclosed herein, treat diseases and disorders associated with elevated triglyceride (TG) levels, such as familial chylomicronemia syndrome (FCS), hypertriglyceridemia, obesity, dyslipidemia, non-alcoholic steatohepatitis, non-alcoholic fatty liver disease, hyperlipidemia, abnormal lipid and/or cholesterol metabolism, atherosclerosis, cardiovascular disease, coronary artery disease, hypertriglyceridemia induced pancreatitis, metabolic syndrome, type II diabetes mellitus, chylomicronemia, multifactorial chylomicronemia, or lipodystrophy syndromes including familial partial lipodystrophy.

### CROSS REFERENCE TO RELATED APPLICATIONS

This application is a continuation of International Patent Application Ser. No. PCT/US20/44879, with an international filing date of 4 Aug. 2020, designating the United States, which claims priority to U.S. Provisional Patent Application Ser. No. 62/883,046, filed on 5 Aug. 2019, U.S. Provisional Patent Application Ser. No. 62/936,559, filed on 17 Nov. 2019, and U.S. Provisional Patent Application Ser. No. 62/970,613, filed on 5 Feb. 2020, the contents of each of which are incorporated herein by reference in their entirety.

### SEQUENCE LISTING

This application contains a Sequence Listing which has been submitted in ASCII format and is hereby incorporated by reference in its entirety. The ASCII copy is named 30683_US1_SequenceListing.txt and is 3 kb in size.

### FIELD OF THE INVENTION

Disclosed herein are methods for the treatment of diseases and disorders that can be mediated at least in part by the silencing or inhibition of Apolipoprotein C-III (also called APOC3, apoC-III, APOC-III, and APO C-III) gene expression, using pharmaceutical compositions that include RNA interference (RNAi) agents that inhibit APOC3 gene expression.

### BACKGROUND

Apolipoprotein (also called APOC3, apoC-III, APOC-III, and APO C-III), encoded by the human Apolipoprotein C-III gene, has recently emerged as a promising target for the treatment of diseases associated with hypertriglyceridemia. Elevated serum triglyceride (TG) levels have been identified as an independent risk factor for cardiovascular disease, and as a contributing factor in the development of atherosclerosis. Individuals with severe hypertriglyceridemia (often >1000 mg/dL) are also at risk of recurrent pancreatitis. Triglycerides are primarily transported in the blood as a major component of very low density lipoprotein cholesterol (VLDL-C) and chylomicron particles, which are known as TG-rich lipoproteins. Lipoproteins are composed of a hydrophobic triacylglycerol and cholesteryl ester core, and a hydrophilic outer layer of phospholipids, cholesterol, and apoproteins. APOC3 is one of these apoproteins.

APOC3 is primarily synthesized in the liver and plays an important role in the production, metabolism, and clearance of TG-rich lipoproteins from plasma. Several gain-of-function polymorphisms have been identified in the promoter region of the APOC3 gene, which are postulated to be contributing factors in development of hypertriglyceridemia (See, e.g., Wang, Y., et al., Association of Apolipoprotein C3 Genetic Polymorphisms with the Risk of Ischemic Stroke in the Northern Chinese Han Population, 11 PLoS One e0163910 (2016); Li, Y., et al., Apolipoprotein C3 gene variants and the risk of coronary heart disease: A meta-analysis 9 Meta Gene 104-109 (2016)). Increased APOC3 synthesis in the liver promotes secretion of TG-rich VLDL-C. In addition, over-abundance of APOC3 inhibits the activity of lipoprotein lipase and hepatic lipase, further increasing serum TG levels by delaying the catabolism of TG-rich lipoproteins. Furthermore, elevated APOC3 also delays the hepatic clearance of TG-rich lipoprotein and their remnant particles by interfering with their binding to hepatic receptors.

Several large genetic analysis studies have reported that individuals with loss-of-function mutations of APOC3 exhibit low levels of triglycerides and reduced incidence of cardiovascular disease. In addition, APOC3-deficient individuals also have increased HDL-C compared to individuals without APOC3 mutations. (See, e.g., Bernelot Moens, S. J., et al., Inhibition of ApoCIII: the next PCSK9? 25 Curr Opin Lipidol 418-422 (2014); Saleheen, D., et al., Human knockouts and phenotypic analysis in a cohort with a high rate of consanguinity, 544 Nature 235-239 (2017)).

Currently, hypertriglyceridemia is often treated with one or more of niacin, fibrates, statins, and fish oil in moderate cases; however, in most cases, the reduction in serum TG is modest. Additionally, available therapeutics are often ineffective in patients with monogenic causes of very severe hypertriglyceridemia (such as patients with familial chylomicronemia syndrome (FCS)) because the majority of disease-causing mutations are in lipoprotein lipase (LPL), with mutations in cofactors or LPL interacting proteins apolipoprotein C-II (APOC2), apolipoprotein AV (APOA5), lipase maturation factor 1 (LMFJ), and glycosylphosphatidylinositol anchored high density lipoprotein binding protein 1 (GPIHBP1) observed less frequently. These mutations lead to dysfunctional lipoprotein lipase, but functional lipoprotein lipase is required for optimal response to standard therapies. There is a need for effective therapeutics that can provide a substantial TG lowering effect for the treatment of diseases such as familial chylomicronemia syndrome (FCS), chylomicronemia, multifactorial chylomicronemia, hypertriglyceridemia induced pancreatitis, metabolic syndrome, type II diabetes mellitus, lipodystrophy syndromes including familial partial lipodystrophy, obesity, dyslipidemia, non-alcoholic steatohepatitis, non-alcoholic fatty liver disease, hyperlipidemia, hypertriglyceridemia, abnormal lipid and/or cholesterol metabolism, atherosclerosis, cardiovascular disease, coronary artery disease, and other dyslipidemias and metabolic-related disorders and diseases.

### SUMMARY

Described herein are methods of treating APOC3-related diseases and disorders in a human subject in need thereof, the methods comprising administering to the human subject a pharmaceutical composition that includes the composition described in Table 2 (i.e., APOC3 RNAi Drug Substance, also referred to herein as ADS-005) at a dose of between about 1 mg and about 100 mg of the APOC3 RNAi Drug Substance, wherein the pharmaceutical composition is administered subcutaneously and there is about one month between doses (i.e., monthly dosing). In some embodiments, the pharmaceutical composition used in the methods disclosed herein comprises, consists of, or consists essentially of the Formulated APOC3 RNAi Drug Substance as described in Table 3 (also referred to herein as ADS-005-1).

The APOC3 RNAi Drug Substance described herein provided unexpected potency and duration of effect. As shown in the data from the human clinical study presented herein, the APOC3 RNAi Drug Substance embodiment in just a single dose of less than 100 mg provided durable inhibition of APOC3 gene expression that lasted several months and resulted substantial reductions in triglycerides (TG) and other relevant lipid parameters, while maintaining a favorable safety profile. This led to the novel discovery of a low-dose and infrequent dosing regimen for the treatment of APOC3-related diseases and disorders.

The methods of treatment described herein lead to a reduction in APOC3 expression in the human subject, which thereby results in a reduction of, among other things, serum triglyceride (TG) levels in the subject.

Additionally, described herein are methods of treating an APOC3-related disease or disorder in a human subject in need thereof, the methods comprising administering to the human subject a pharmaceutical composition that includes the APOC3 RNAi Drug Substance as described in Table 2 (i.e., ADS-005) at a dose of between about 1 mg and about 50 mg, wherein the pharmaceutical composition is administered subcutaneously and there is about one month between dose administrations (i.e., monthly dosing).

Further described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 100 mg, wherein the pharmaceutical composition is administered subcutaneously and there is about three months between dose administrations (e.g., quarterly dosing or dosing every 12 weeks (q12w)).

Also described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 50 mg, wherein the pharmaceutical composition is administered subcutaneously and there is about three months between dose administrations (e.g., quarterly dosing or dosing every 12 weeks (q12w)).

Described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 100 mg, wherein the pharmaceutical composition is administered subcutaneously, and wherein the initial dose is followed by a second dose about one month later, and thereafter for subsequent doses there is about three months between dose administrations.

Described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 50 mg, wherein the pharmaceutical composition is administered subcutaneously, and wherein the initial dose is followed by a second dose about one month later, and thereafter for subsequent doses there is about three months between dose administrations.

Further described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 100 mg, wherein the pharmaceutical composition is administered subcutaneously and there is about four months between dose administrations.

Also described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 50 mg, wherein the pharmaceutical composition is administered subcutaneously and there is about four months between dose administrations.

Described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 100 mg, wherein the pharmaceutical composition is administered subcutaneously, and wherein the initial dose is followed by a second dose about one month later, and thereafter for subsequent doses there is about four months between dose administrations.

Described herein are methods of treating an APOC3-related disease or disorder in a human subject in need thereof, the methods comprising administering to the human subject a pharmaceutical composition that includes the APOC3 RNAi Drug Substance as described in Table 2 at a dose of between about 1 mg and about 50 mg, wherein the pharmaceutical composition is administered subcutaneously, and wherein the initial dose is followed by a second dose about one month later, and thereafter for subsequent doses there is about four months between dose administrations.

In some embodiments, the dose of APOC3 RNAi Drug Substance administered in each dose is between about 10 mg and about 100 mg. In some embodiments, the dose of APOC3 RNAi Drug Substance administered in each dose is between about 10 mg and about 50 mg. In some embodiments, the dose of APOC3 RNAi Drug Substance administered in each dose is between about 25 mg and about 50 mg. In some embodiments, the dose of APOC3 RNAi Drug Substance administered in each dose is about 25 mg. In some embodiments, the dose of APOC3 RNAi Drug Substance administered in each dose is about 50 mg. In some embodiments, the dose of APOC3 RNAi Drug Substance administered in each dose is about 100 mg. In some embodiments, the dose of APOC3 RNAi Drug Substance administered in each dose is about 10 mg. In some embodiments, the dose of APOC3 RNAi Drug Substance administered in each dose is no greater than 100 mg. In some embodiments, the dose of APOC3 RNAi Drug Substance administered in each dose is no greater than 50 mg. In some embodiments, the dose of APOC3 RNAi Drug Substance administered in each dose is no greater than 25 mg.

The treatment methods disclosed herein can provide a substantial TG lowering effect for the treatment of APOC3-related diseases and disorders such as hypertriglyceridemia induced pancreatitis, metabolic syndrome, type II diabetes mellitus, familial chylomicronemia syndrome (FCS), chylomicronemia, multifactorial chylomicronemia, lipodystrophy syndromes including familial partial lipodystrophy, obesity, dyslipidemia, non-alcoholic steatohepatitis, non-alcoholic fatty liver disease, hyperlipidemia, hypertriglyceridemia, abnormal lipid and/or cholesterol metabolism, atherosclerosis, cardiovascular disease, coronary artery disease, and other dyslipidemias and metabolic-related disorders and diseases. The methods disclosed herein can, in some embodiments, treat the APOC3-related diseases or disorders by substantially lower TG levels and thereby reducing the risk of developing hypertriglyceridemia induced pancreatitis, metabolic syndrome, type II diabetes mellitus, familial chylomicronemia syndrome (FCS), chylomicronemia, multifactorial chylomicronemia, lipodystrophy syndromes including familial partial lipodystrophy, obesity, dyslipidemia, non-alcoholic steatohepatitis, non-alcoholic fatty liver disease, hyperlipidemia, hypertriglyceridemia, abnormal lipid and/or cholesterol metabolism, atherosclerosis, cardiovascular disease, coronary artery disease, or other dyslipidemias or metabolic-related disorders and diseases.

The pharmaceutical compositions that include APOC3 RNAi agents disclosed herein can be administered to a human subject to inhibit the expression of an APOC3 gene in the subject. In some embodiments, the subject is a human that has been previously diagnosed with having elevated triglyceride levels, over-expression of APOC3 protein, or one or more APOC3-related diseases or disorders.

Other objects, features, aspects, and advantages of the invention will be apparent from the following detailed description, accompanying figures, and from the claims.

### BRIEF DESCRIPTION OF THE DRAWINGS

FIG. 1A to 1D. Chemical structure representation of APOC3 RNAi Drug Substance described in Table 2 (referred to herein as ADS-005; i.e., APOC3 RNAi agent conjugated to a tridentate N-acetyl-galactosamine targeting group at the 5′ terminal end of the sense strand), shown in a free acid form.

FIG. 2A to 2D. Chemical structure representation of APOC3 RNAi Drug Substance described in Table 2, shown in a sodium salt form.

FIG. 3. Schematic diagram of the modified sense and antisense strands of APOC3 RNAi Drug Substance described in Table 2 (referred to herein as ADS-005; i.e., APOC3 RNAi agent conjugated to a tridentate N-acetyl-galactosamine targeting group at the 5′ terminal end of the sense strand). The following abbreviations are used in FIG. 3: a, c, g, i, and u are 2′-O-methyl modified nucleotides (i represents inosine); Af, Cf, Gf, and Uf are 2′-fluoro (also referred to in the art as 2′-deoxy-2′-fluoro) modified nucleotides; o is a phosphodiester linkage; s is a phosphorothioate linkage; invAb is an inverted abasic residue or subunit; and (NAG37)s is a tridentate N-acetyl-galactosamine targeting ligand having the following chemical structure:

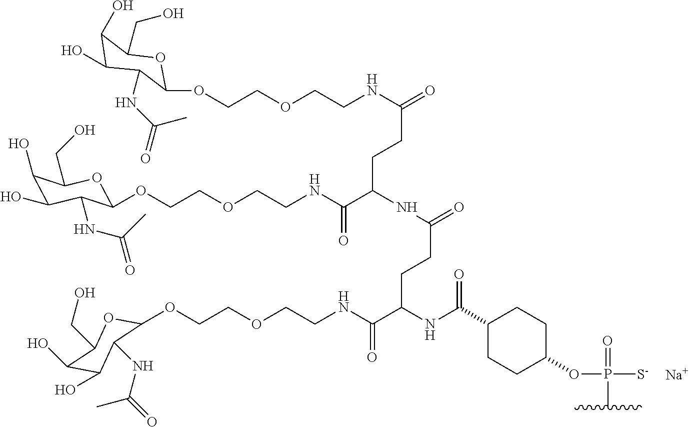

(shown in sodium salt form),

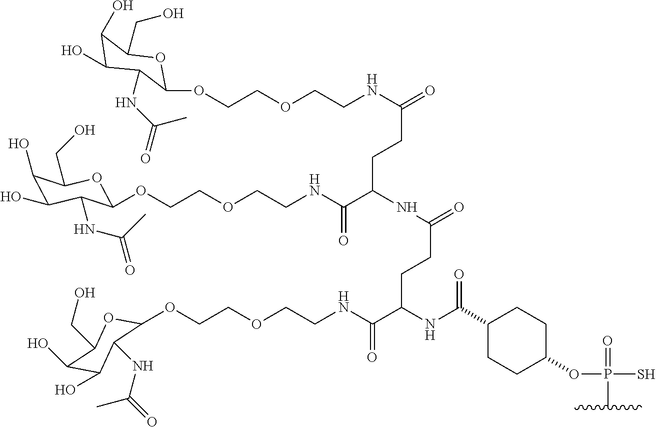

(shown in free acid form).

FIG. 4. Phase I study design and dose escalation schedule (as amended) for the Phase I clinical study described in Example 2.

FIG. 5. Summary of Cohorts for the Phase I clinical study (as amended) described in Example 2.

FIG. 6. Graph showing serum APOC3 protein levels in healthy human volunteers administered with placebo (all Cohorts) or 25 mg of APOC3 RNAi Drug Substance (Cohort 1) from the Phase I clinical study described in Example 2. As shown in FIGS. 6 through 16, the number of mg shown in the graph for each respective Figure refers to the amount dosed of the APOC3 RNAi Drug Substance described in Table 2 (administered as Formulated APOC3 RNAi Drug Substance as described in Table 3).

FIG. 7. Graph showing serum APOC3 protein levels in healthy human volunteers administered with placebo (all Cohorts) or a single 50 mg dose of APOC3 RNAi Drug Substance (Cohort 2) from the Phase I clinical study described in Example 2.

FIG. 8. Graph showing serum APOC3 protein levels in healthy human volunteers administered with placebo (all Cohorts) or a single 100 mg dose of APOC3 RNAi Drug Substance (Cohort 3) from the Phase I clinical study described in Example 2.

FIG. 9. Graph showing serum APOC3 protein levels in healthy human volunteers administered with placebo (all Cohorts) or a single 10 mg dose of APOC3 RNAi Drug Substance (Cohort 3) from the Phase I clinical study described in Example 2.

FIG. 10. Graph showing serum triglyceride (TG) levels in healthy human volunteers administered with placebo (all Cohorts) or a single 25 mg dose of APOC3 RNAi Drug Substance (Cohort 1) from the Phase I clinical study described in Example 2.

FIG. 11. Graph showing serum triglyceride (TG) levels in healthy human volunteers administered with placebo (all Cohorts) or a single 50 mg dose of APOC3 RNAi Drug Substance (Cohort 2) from the Phase I clinical study described in Example 2.

FIG. 12. Graph showing serum triglyceride (TG) levels in healthy human volunteers administered with placebo (all Cohorts) or a single 100 mg dose of APOC3 RNAi Drug Substance (Cohort 3) from the Phase I clinical study described in Example 2.

FIG. 13. Graph showing serum triglyceride (TG) levels in healthy human volunteers administered with placebo (all Cohorts) or a single 10 mg dose of APOC3 RNAi Drug Substance (Cohort 4) from the Phase I clinical study described in Example 2.

FIG. 14. Graph showing serum very low density lipoprotein cholesterol (VLDL-C) levels in healthy human volunteers administered with placebo (all Cohorts) or a single 25 mg dose of APOC3 RNAi Drug Substance (Cohort 1) from the Phase I clinical study described in Example 2.

FIG. 15. Graph showing serum VLDL-C levels in healthy human volunteers administered with placebo (all Cohorts) or a single 50 mg dose of APOC3 RNAi Drug Substance (Cohort 2) from the Phase I clinical study described in Example 2.

FIG. 16. Graph showing serum VLDL-C levels in healthy human volunteers administered with placebo (all Cohorts) or a single 100 mg dose of APOC3 RNAi Drug Substance (Cohort 3) from the Phase I clinical study described in Example 2.

FIG. 17. Graph showing serum VLDL-C levels in healthy human volunteers administered with placebo (all Cohorts) or a single 10 mg dose of APOC3 RNAi Drug Substance (Cohort 4) from the Phase I clinical study described in Example 2.

FIG. 18. Graph showing serum very high density lipoprotein cholesterol (HDL-C) levels in healthy human volunteers administered with placebo (all Cohorts) or a single 25 mg dose of APOC3 RNAi Drug Substance (Cohort 1) from the Phase I clinical study described in Example 2.

FIG. 19. Graph showing serum HDL-C levels in healthy human volunteers administered with placebo (all Cohorts) or a single 50 mg dose of APOC3 RNAi Drug Substance (Cohort 2) from the Phase I clinical study described in Example 2.

FIG. 20. Graph showing serum HDL-C levels in healthy human volunteers administered with placebo (all Cohorts) or a single 100 mg dose of APOC3 RNAi Drug Substance (Cohort 3) from the Phase I clinical study described in Example 2.

FIG. 21. Graph showing serum HDL-C levels in healthy human volunteers administered with placebo (all Cohorts) or a single 10 mg dose of APOC3 RNAi Drug Substance (Cohort 4) from the Phase I clinical study described in Example 2.

FIG. 22. Graph showing serum low density lipoprotein cholesterol (LDL-C) levels in healthy human volunteers administered with placebo (all Cohorts) or a single 25 mg dose of APOC3 RNAi Drug Substance (Cohort 1) from the Phase I clinical study described in Example 2.

FIG. 23. Graph showing serum LDL-C levels in healthy human volunteers administered with placebo (all Cohorts) or a single 50 mg dose of APOC3 RNAi Drug Substance (Cohort 2) from the Phase I clinical study described in Example 2.

FIG. 24. Graph showing serum LDL-C levels in healthy human volunteers administered with placebo (all Cohorts) or a single 100 mg dose of APOC3 RNAi Drug Substance (Cohort 3) from the Phase I clinical study described in Example 2.

FIG. 25. Graph showing serum LDL-C levels in healthy human volunteers administered with placebo (all Cohorts) or a single 10 mg dose of APOC3 RNAi Drug Substance (Cohort 4) from the Phase I clinical study described in Example 2.

FIG. 26. Graph showing serum APOC3 protein levels in chylomicronemia patients administered 50 mg dose of APOC3 RNAi Drug Substance (Cohort 5) from the Phase I clinical study described in Example 2.

FIG. 27. Graph showing serum triglyceride levels in chylomicronemia patients administered 50 mg dose of APOC3 RNAi Drug Substance (Cohort 5) from the Phase I clinical study described in Example 2.

### DETAILED DESCRIPTION

### RNAi Agents

The methods described herein include the administration of a pharmaceutical composition to a human subject, wherein the pharmaceutical composition includes a composition that contains an RNA interference (RNAi) agent (referred to herein and in the art as an RNAi agent or an RNAi trigger) capable of inhibiting expression of an APOC3 gene. In some embodiments, the methods described herein include the administration of a pharmaceutical composition to a human subject, wherein the pharmaceutical composition includes the APOC3 RNAi Drug Substance described in Table 2 (also referred to as ADS-005). The compositions suitable for use in the methods disclosed herein are comprised of an RNAi agent that inhibits expression of an APOC 3 gene in a human subject, and a targeting moiety or targeting group. In some embodiments, the RNAi agent includes the nucleotide sequences provided in Table 1A and 1B, and the sense strand of the RNAi agent is further linked or conjugated to a targeting group comprising three N-acetyl-galactosamine targeting moieties (see, e.g., Table B). An RNAi agent that inhibits expression of an APOC3 gene in a human subject is referred to as an “APOC3 RNAi agent.”

In general, APOC3 RNAi agents comprise a sense strand (also referred to as a passenger strand) and an antisense strand (also referred to as a guide strand) that are annealed to form a duplex. The APOC3 RNAi agents disclosed herein include an RNA or RNA-like (e.g., chemically modified RNA) oligonucleotide molecule capable of degrading or inhibiting translation of messenger RNA (mRNA) transcripts of APOC3 mRNA in a sequence specific manner. The APOC3 RNAi agents disclosed herein may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that the APOC3 RNAi agents, as that term is used herein, operate primarily through the RNA interference mechanism, the disclosed RNAi agents are not bound by or limited to any particular pathway or mechanism of action. RNAi agents in general are comprised of a sense strand and an antisense strand that are each 16 to 49 nucleotides in length, and include, but are not limited to: short or small interfering RNAs (siRNAs), double-strand RNAs (dsRNA), micro RNAs (miRNAs), short hairpin RNAs (shRNA), and dicer substrates.

The length of an APOC3 RNAi agent sense strand is typically 16 to 49 nucleotides in length, and the length of an APOC3 RNAi agent antisense strand is typically 18 to 49 nucleotides in length. In some embodiments, the sense and antisense strands are independently 17 to 26 nucleotides in length. In some embodiments, the sense and antisense strands are independently 21 to 26 nucleotides in length. In some embodiments, the sense and antisense strands are independently 21 to 24 nucleotides in length. In some embodiments, the sense and/or antisense strands are independently 16, 17, 18, 19, 20, 21, 22, 23, 24, 25, 26, 27, 28, 29, or 30 nucleotides in length. In some embodiments, the sense strand and the antisense strand are both 21 nucleotides in length. The sense and antisense strands can be either the same length or different lengths. The sense and antisense strands can also form overhanging nucleotides on one or both ends of the APOC3 RNAi agent.

APOC3 RNAi agents inhibit, silence, or knockdown APOC3 gene expression. As used herein, the terms “silence,” “reduce,” “inhibit,” “down-regulate,” or “knockdown,” when referring to expression of APOC3, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agent as compared to a second cell, group of cells, tissue, organ, or subject that has not or have not been so treated. In some instances, the reduction in gene expression is measured by comparing the baseline levels of APOC3 mRNA or APOC3 protein in a human subject prior to administration of a composition that comprises an APOC3 RNAi agent, with the APOC3 mRNA or APOC3 protein levels after administration of the therapeutic.

APOC3 gene inhibition, silencing, or knockdown may be measured by any appropriate assay or method known in the art. The non-limiting Examples set forth herein, as well as the examples set forth in International Patent Application Publication No. WO 2019/051402 (Patent Application No. PCT/US2018/050248), which is incorporated by reference herein in its entirety, provide certain examples of appropriate assays for measuring APOC3 gene expression inhibition. A reference APOC3 mRNA gene transcript for normal humans (referred to as transcript variant 1; GenBank NM_000040.1) can be found at SEQ ID NO:1.

APOC3 RNAi agents suitable for use in the methods disclosed herein can be covalently linked or conjugated to a targeting group that includes one or more N-acetyl-galactosamine moieties. In some embodiments, APOC3 RNAi agents suitable for use in the methods disclosed herein are covalently linked or conjugated to a targeting group that includes one or more N-acetyl-galactosamine moieties thereby forming the APOC3 RNAi Drug Substance described in Table 2. In some embodiments, the methods described herein include the administration of the APOC3 RNAi Drug Substance described in Table 2. The APOC3 RNAi Drug Substance described in Table 2 includes the APOC3 RNAi agent shown in Table 1A (antisense strand) and Table 1B (sense strand). The N-acetyl-galactosamine moieties facilitate the targeting of the APOC3 RNAi agent to the asialoglycoprotein receptors (ASGPr) readily present on the surface of hepatocytes, which leads to internalization of the APOC3 RNAi agent by endocytosis or other means.

The APOC3 RNAi agents that can be suitable for use in the methods disclosed herein include an antisense strand that has a region of complementarity to at least a portion of an APOC3 mRNA. APOC3 RNAi agents and APOC3 RNAi Drug Substances suitable for use in the disclosed methods are described in International Patent Application Publication No. WO 2019/051402 (Patent Application No. PCT/US2018/050248), which as previously noted is incorporated by reference herein in its entirety.

As used herein, the terms “sequence” and “nucleotide sequence” mean a succession or order of nucleobases or nucleotides, described with a succession of letters using standard nomenclature. As used herein, the terms “nucleobase” and “nucleotide” have the same meaning as commonly understood in the art.

As used herein, the term “complementary,” when used to describe a first nucleotide sequence (e.g., RNAi agent antisense strand) in relation to a second nucleotide sequence (e.g., RNAi agent sense strand or targeted mRNA sequence), means the ability of an oligonucleotide that includes the first nucleotide sequence to hybridize (form base pair hydrogen bonds under mammalian physiological conditions (or otherwise suitable conditions) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that all (100%) of the bases in a contiguous sequence of a first oligonucleotide will hybridize with the same number of nucleotides in a contiguous sequence of a second oligonucleotide. The contiguous sequence may comprise all or a part of a first or second nucleotide sequence.

As used herein, “partially complementary” means that in a hybridized pair of nucleotide sequences, at least 70%, but not all, of the bases in a contiguous sequence of a first oligonucleotide will hybridize with the same number of bases in a contiguous sequence of a second polynucleotide.

As used herein, “substantially complementary” means that in a hybridized pair of nucleotide sequences, at least 85%, but not all, of the bases in a contiguous sequence of a first oligonucleotide will hybridize with the same number of bases in a contiguous sequence of a second polynucleotide. The terms “complementary,” “fully complementary,” “partially complementary,” and “substantially complementary” herein are used with respect to the nucleotide matching between the sense strand and the antisense strand of an RNAi agent, or between the antisense strand of an RNAi agent and a sequence of an APOC3 mRNA.

As used herein, the term “substantially identical” or “substantially identity” as applied to nucleic acid sequence means that a nucleic acid sequence comprises a sequence that has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

### Modified Nucleotides and Modified Internucleoside Linkages

The APOC3 RNAi agents disclosed herein can be comprised of modified nucleotides, which can preserve activity of the RNAi agent while at the same time increasing the serum stability, as well as minimize the possibility of activating interferon activity in humans. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include any known modified nucleotides known in the art, including but not limited to, deoxyribonucleotides, nucleotide mimics, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-arabino nucleotides, 5′-Me, 2′-fluoro nucleotides, morpholino nucleotides, vinyl phosphonate-containing nucleotides, and cyclopropyl phosphonate-containing nucleotides. In some embodiments, the modified nucleotides of an APOC3 RNAi agent are 2′-modified nucleotides (i.e. a nucleotide with a group other than a hydroxyl group at the 2′ position of the five-membered sugar ring). 2′-modified nucleotides include, but are not limited to, 2′-O-methyl nucleotides, 2′-deoxy-2′-fluoro nucleotides (commonly referred to simply as 2′-Fluoro nucleotides), 2′-deoxy nucleotides, 2′-methoxyethyl (2′-O-2-methoxyethyl) nucleotides, 2′-amino nucleotides, and 2′-alkyl nucleotides. Additional 2′-modified nucleotides are known in the art. It is not necessary for all nucleotides in a given RNAi agent to be uniformly modified. Additionally, more than one modification can be incorporated in a single APOC3 RNAi agent or even in a single nucleotide thereof. The APOC3 RNAi agent sense strands and antisense strands can be synthesized and/or modified by methods known in the art. Modification at one nucleotide is independent of modification at another nucleotide.

In some embodiments, the nucleobase (often referred to as simply the “base”) may be modified. As is commonly used in the art, natural nucleobases include the primary purine bases adenine and guanine, and the primary pyrimidine bases cytosine, thymine, and uracil. A nucleobase may be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

Modified nucleobases include, for example, 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxy methyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an APOC3 RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides.

In some embodiments, one or more nucleotides of an APOC3 RNAi agent are linked by non-standard linkages or backbones (i.e., modified internucleoside linkages or modified backbones). Modified internucleoside linkages or backbones include, but are not limited to, phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an APOC3 RNAi agent can contain 1, 2, 3, 4, 5, or 6 phosphorothioate linkages, an antisense strand of an APOC3 RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an APOC3 RNAi agent can contain 1, 2, 3, or 4 phosphorothioate linkages, an antisense strand of an APOC3 RNAi agent can contain 1, 2, 3, or 4 phosphorothioate linkages, or both the sense strand and the antisense strand independently can contain 1, 2, 3, or 4 phosphorothioate linkages.

In some embodiments, an APOC3 RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, phosphorothioate internucleoside linkages are used to link the terminal nucleotides in the sense strand to capping residues present at the 5′-end, the 3′-end, or both the 5′- and 3′-ends of the nucleotide sequence. In some embodiments, phosphorothioate internucleoside linkages are used to link a targeting group to the sense strand.

In some embodiments, an APOC3 RNAi agent antisense strand contains three or four phosphorothioate internucleoside linkages. In some embodiments, an APOC3 RNAi agent antisense strand contains three phosphorothioate internucleoside linkages. In some embodiments, the three phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, an APOC3 RNAi agent contains at least two phosphorothioate internucleoside linkages in the sense strand and three or four phosphorothioate internucleoside linkages in the antisense strand.

In some embodiments, an APOC3 RNAi agent contains one or more modified nucleotides and one or more modified internucleoside linkages. In some embodiments, a 2′-modified nucleoside is combined with modified internucleoside linkage.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table A). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

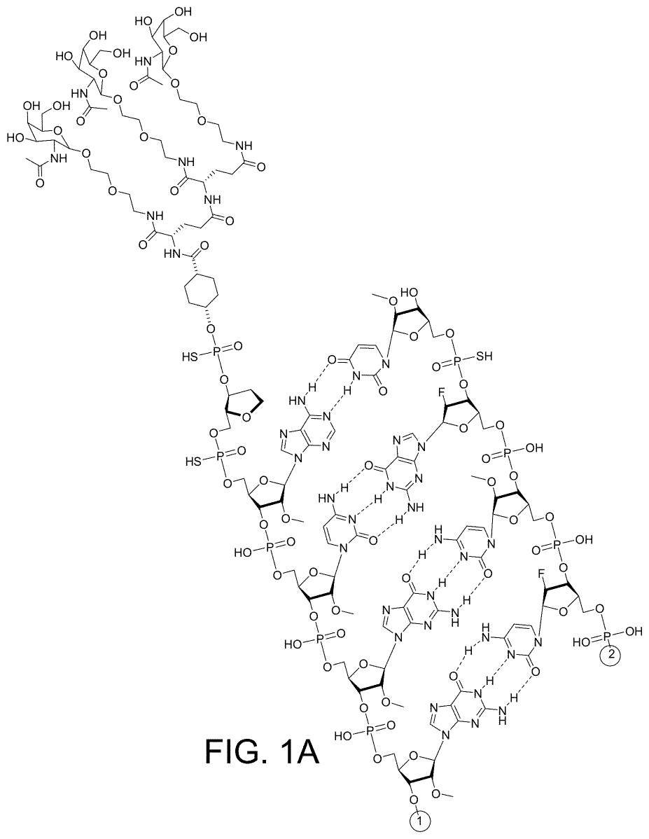

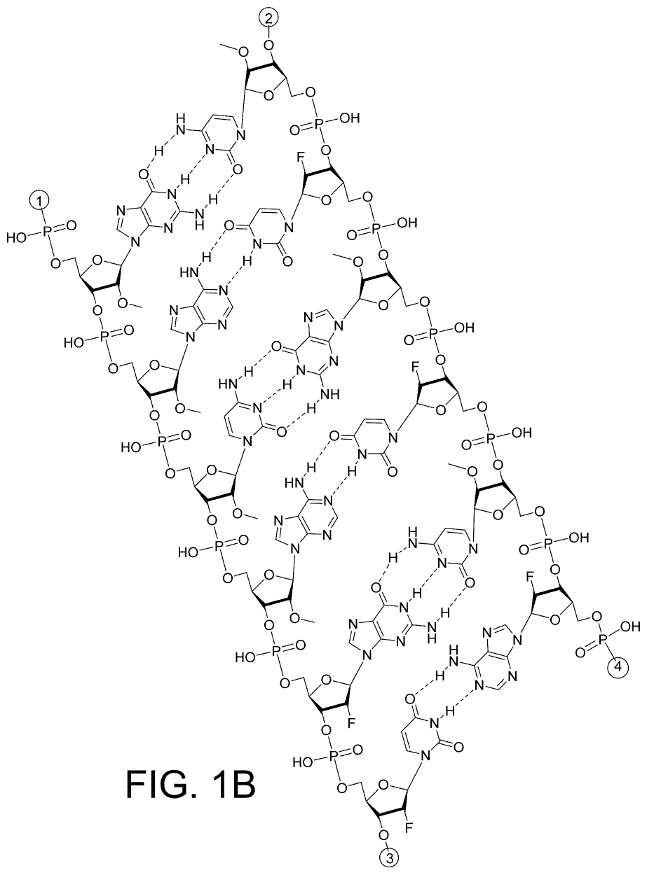

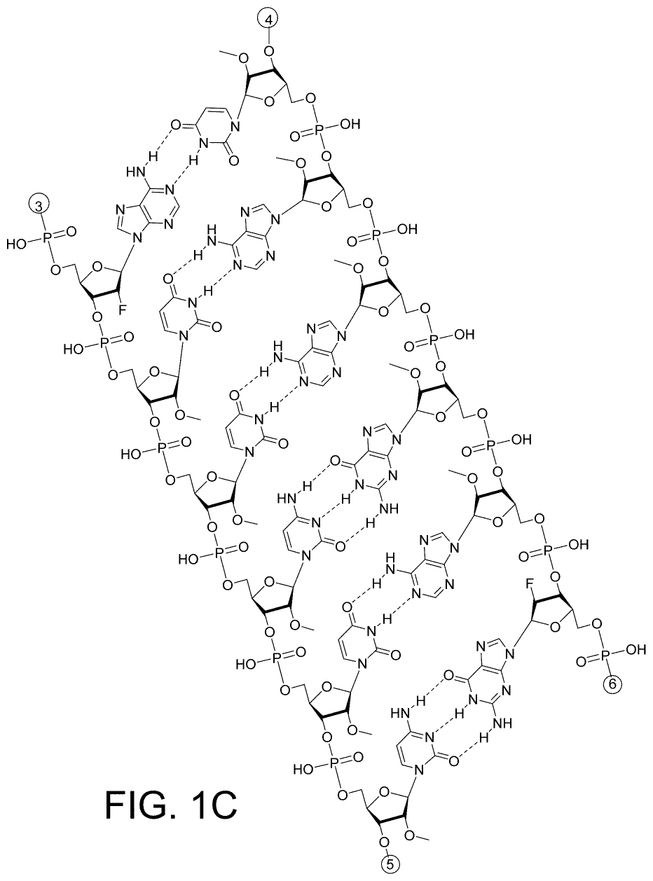

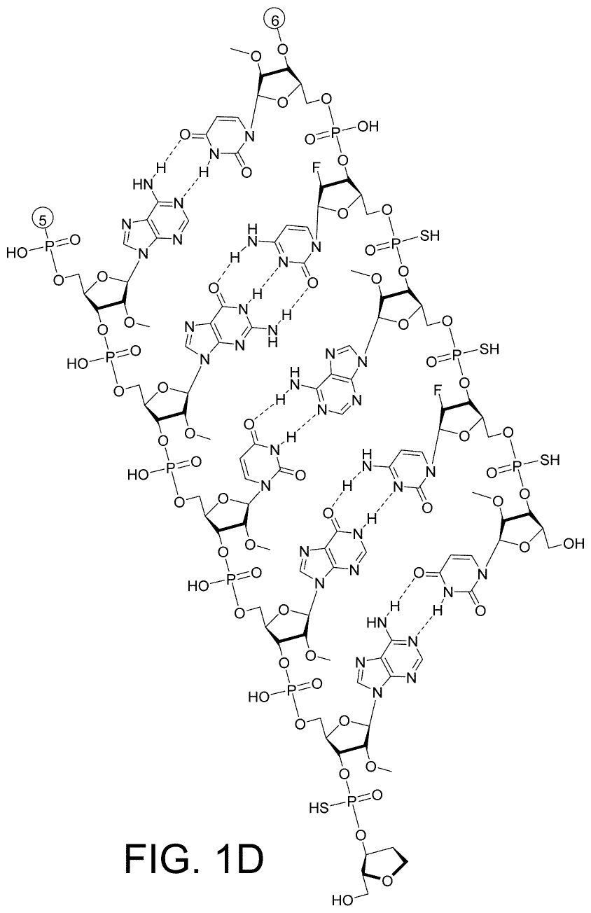

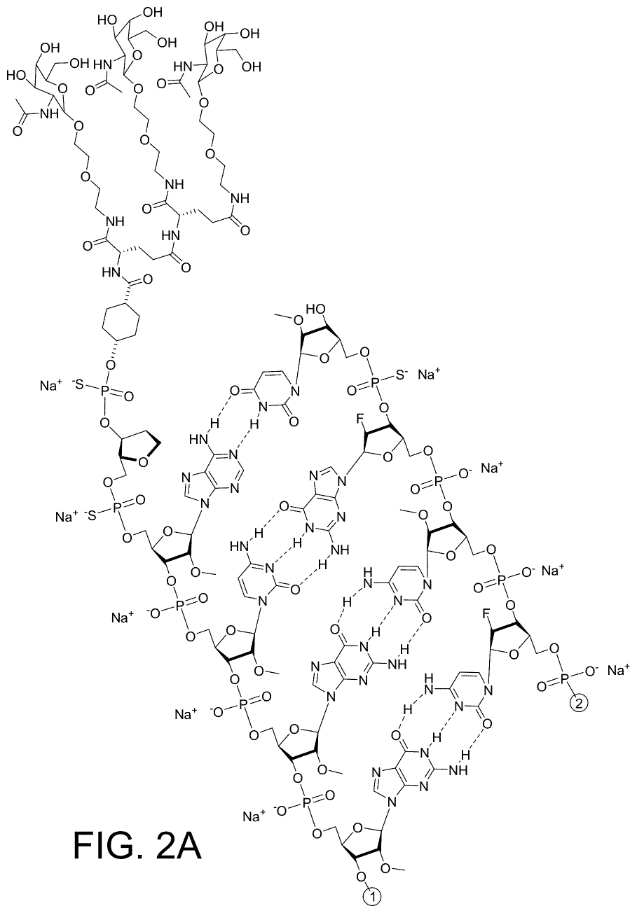

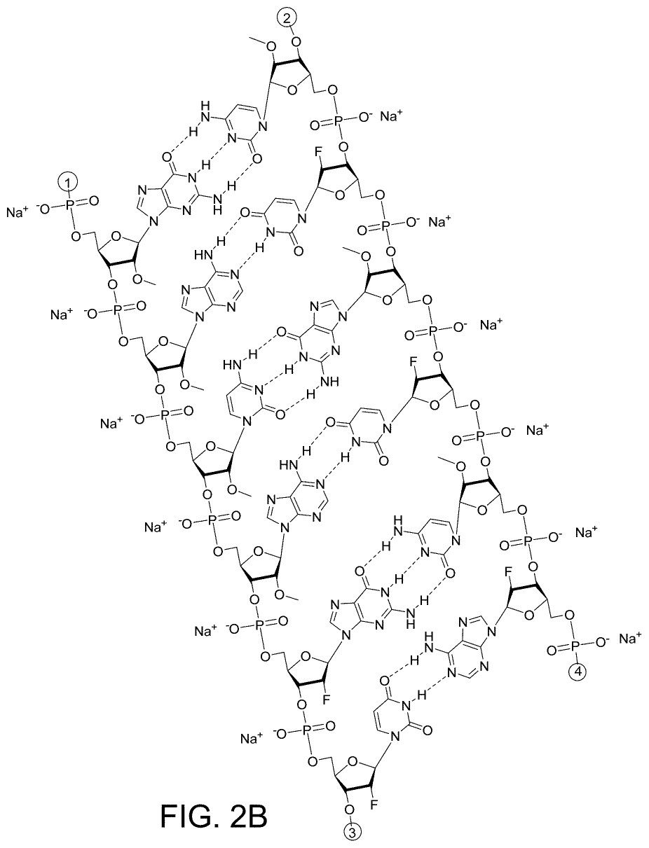

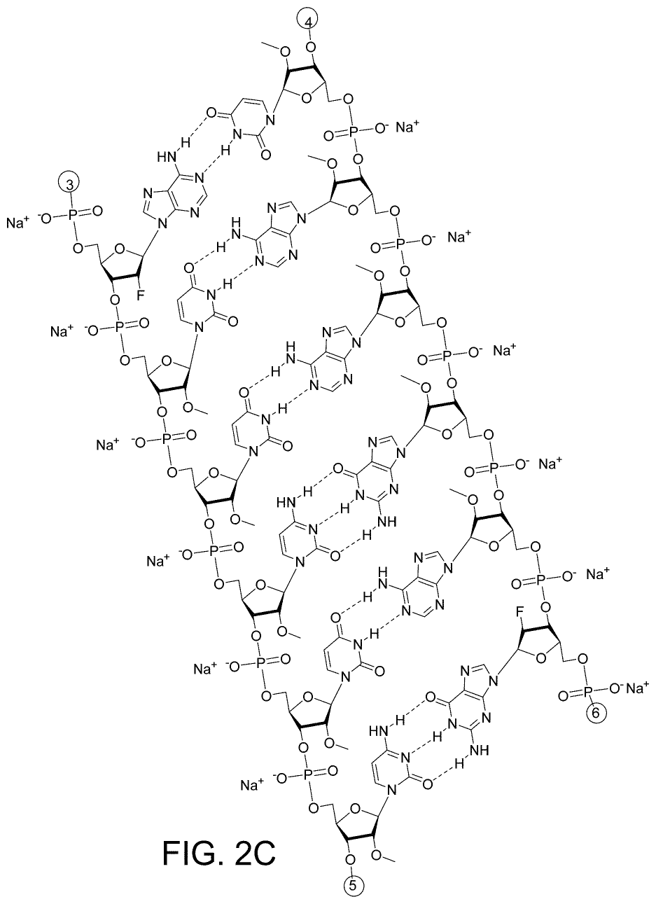

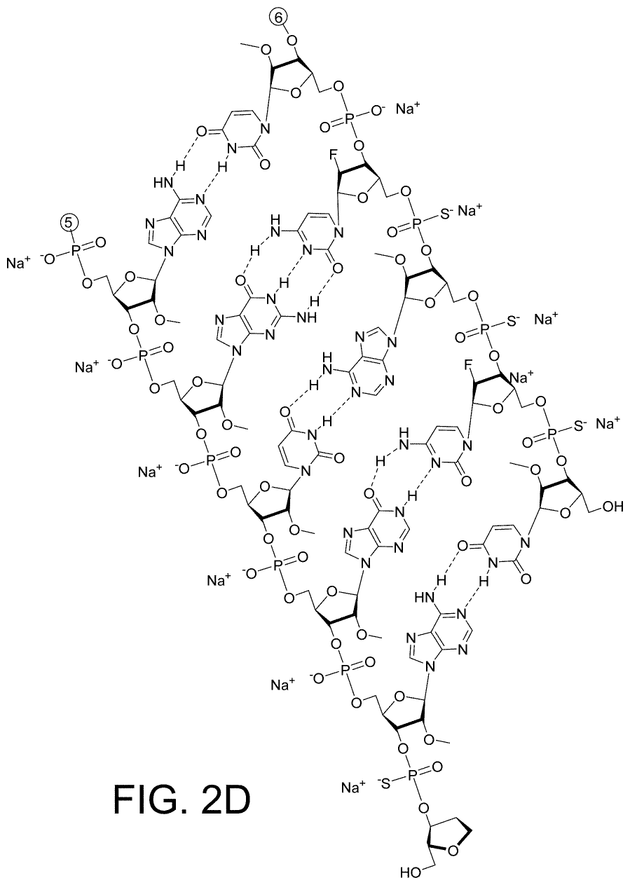

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other internucleoside linkages. The chemical structures for inverted abasic deoxyribose residues are shown in Table A below, as well as in the chemical structures shown in FIGS. 1A to 1D and FIGS. 2A to 2D.

TABLE A
Inverted Abasic (Deoxyribose) Chemical Structures

When positioned internally on oligonucleotide:

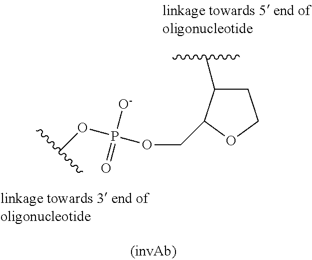 [SMILES: *OP(=O)([O-])OCC1OCCC1*]

When positioned internally on oligonucleotide:

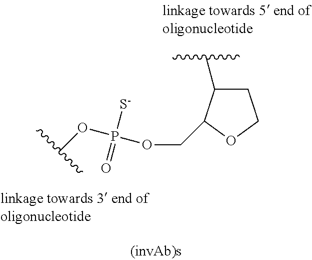 [SMILES: *OP(=O)([S-])OCC1OCCC1*]

When positioned at the 3′ terminal end of oligonucleotide:

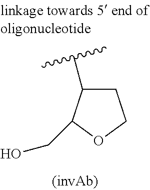 [SMILES: *C1CCOC1CO]

### Targeting Moieties and Groups

An APOC3 RNAi agent can be conjugated to one or more non-nucleotide groups including, but not limited to, a targeting moiety or a targeting group. A targeting moiety or targeting group can enhance targeting or delivery of the RNAi agent. Examples of targeting moieties and targeting groups are known in the art. Specific examples of the (NAG37)s targeting group used in the APOC3 RNAi Drug Substance described in Table 2 herein, which includes three N-acetyl-galactosamine targeting moieties, is provided in Table B. The targeting moiety or targeting group can be covalently linked to the 3′ and/or 5′ end of either the sense strand and/or the antisense strand. In some embodiments, an APOC3 RNAi agent contains a targeting group linked to the 3′ and/or 5′ end of the sense strand. In some embodiments, a targeting group is linked to the 5′ end of an APOC3 RNAi agent sense strand. In some embodiments, the targeting group comprises, consists essential of, or consists of the structure (NAG37)s, and is linked to the 5′ end of an APOC3 RNAi agent sense strand. A targeting group can be linked directly or indirectly to the RNAi agent via a linker/linking group. In some embodiments, a targeting group is linked to the RNAi agent via a labile, cleavable, or reversible bond or linker. In some embodiments, a targeting group is linked to an inverted abasic residue at the 5′ end of the sense strand.

Targeting groups or targeting moieties can enhance the pharmacokinetic or biodistribution properties of a conjugate or RNAi agent to which they are attached to improve cell-specific distribution and cell-specific uptake of the conjugate or RNAi agent. In some embodiments, a targeting group enhances endocytosis of the RNAi agent. A targeting group can be monovalent, divalent, trivalent, tetravalent, or have higher valency for the target to which it is directed. Representative targeting groups include, without limitation, compounds with affinity to cell surface molecules, cell receptor ligands, haptens, antibodies, monoclonal antibodies, antibody fragments, and antibody mimics with affinity to cell surface molecules.

In some embodiments, a targeting group comprises an asialoglycoprotein receptor ligand. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formylgalactosamine, N-acetyl-galactosamine, N-propionyl-galactosamine, N-n-butanoyl-galactosamine, and N-iso-butanoyl-galactosamine (see for example: S. T. Iobst and K. Drickamer, J. B. C., 1996, 271, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945).

Galactose derivatives have been used to target molecules to hepatocytes in vivo through their binding to the asialoglycoprotein receptor expressed on the surface of hepatocytes. Binding of asialoglycoprotein receptor ligands to the asialoglycoprotein receptor(s) facilitates cell-specific targeting to hepatocytes and endocytosis of the molecule into hepatocytes. Asialoglycoprotein receptor ligands can be monomeric (e.g., having a single galactose derivative) or multimeric (e.g., having multiple galactose derivatives). The galactose derivative or galactose derivative “cluster” can be attached to the 3′ or 5′ end of the sense or antisense strand of the RNAi agent using methods known in the art.

In some embodiments, a targeting group comprises a galactose derivative cluster. As used herein, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamines. In some embodiments, the galactose derivative cluster comprises three N-acetyl-galactosamines. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamines.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see, for example, U.S. Pat. No. 5,885,968; Biessen et al. J. Med. Chem. 1995 Vol. 39 p. 1538-1546). The branch point can be any small molecule which permits attachment of three galactose derivatives and further permits attachment of the branch point to the RNAi agent. An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative cluster is comprised of a galactose derivative tetramer, which can be, for example, an N-acetyl-galactosamine tetramer.

The preparation of targeting groups, such as galactose derivative clusters that include N-acetyl-galactosamine, is described in, for example, International Patent Application Publication No. WO 2018/044350 (Patent Application No. PCT/US2017/021147) and International Patent Application Publication No. WO 2017/156012 (Patent Application No. PCT/US2017/021175), the contents of both of which are incorporated by reference herein in their entirety.

For example, the targeting ligand conjugated to the APOC3 RNAi agent described in Tables 1A and 1B has the chemical structure of (NAG37)s, as shown in the following Table B.

TABLE B
Chemical Structure of (NAG37)s.

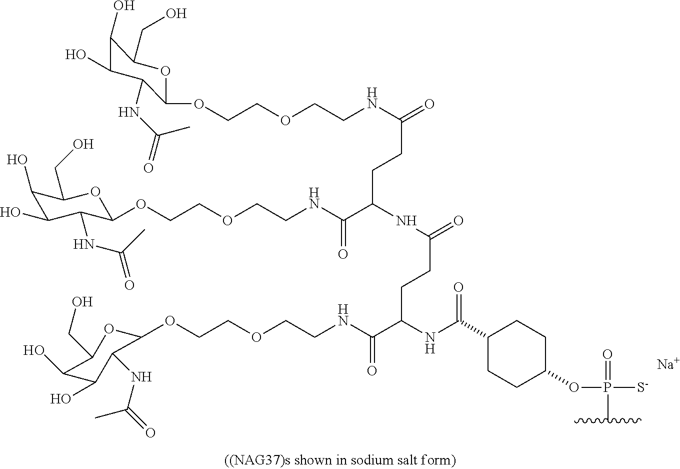

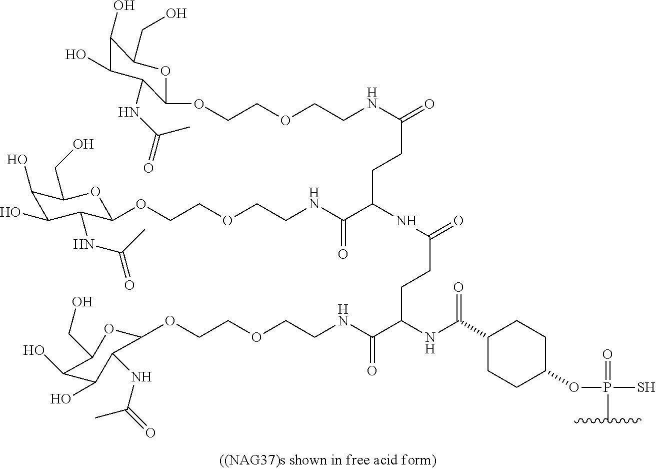

### APOC3 RNAi Agents and APOC3 RNAi Drug Substance (ADS-005)

In some embodiments, the APOC3 RNAi agent used in the methods disclosed herein have the nucleotide sequences of the APOC3 RNAi Drug Substance (ADS-005) shown in Table 2. The nucleotide sequences of the APOC3 RNAi agent found in APOC3 RNAi Drug Substance include an antisense strand nucleotide sequence as set forth in the following Table 1A, and a sense strand nucleotide sequence as set forth in the following Table 1B.

TABLE 1A
APOC3 RNAi Agent Ant.sense Strand Sequence

| SEQ | Antisense Sequence | SEQ | Underlying |
|---|---|---|---|
| ID | (Modified) | ID | Base Sequence |
| NO. | (5′ → 3′) | NO. | (5′ → 3′) |
| 2 | usCfsasCfuGfagaau | 3 | UCACUGAGAAUA |
| | AfcUfgUfcCfcGfsu | | CUGUCCCGU |

TABLE 1B
APOC3 RNAi Agent Sense Strand Nucleotide
Sequence (shown as modified version without
inverted abasic residues or NAG targeting
group present in APOC3 RNAi Drug Substance)

| | | | Underlying |
|---|---|---|---|
| SEQ | Sense Sequence | SEQ | Base |
| ID | (Modified) | ID | Sequence |
| NO. | (5′ → 3′) | NO. | (5′ → 3′) |
| 4 | acgggacaGfUf | 5 | ACGGGACAGU |
| | Afuucucaguia | | AUUCUCAGUIA |

As used in Tables 1A, 1B, and 2 herein, the following notations are used to indicate modified nucleotides, targeting groups, and linking groups: A, C, G, 1, and U represent adenosine, cytidine, guanosine, inosine, and uridine, respectively; a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue (see Table A); and (NAG37)s represents the structure shown in Table B, above.

As the person of ordinary skill in the art would readily understand, unless otherwise indicated by the sequence (such as, for example, by a phosphorothioate linkage “s”), when present in a strand, the monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 1-3 showing all linkages). Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand. Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the APOC3 RNAi agents and compositions that include APOC3 RNAi agents disclosed herein.

Each sense strand and/or antisense strand can have any targeting groups or linking groups listed above, as well as other targeting or linking groups, conjugated to the 5′ and/or 3′ end of the sequence.

The APOC3 RNAi agent antisense strand sequence is designed to target mRNA transcripts from an APOC3 gene in a human subject, thereby silencing translation of APOC3 protein using an RNA interference mechanism for human subjects with APOC3.

In some embodiments, the methods disclosed herein use the APOC3 RNAi Drug Substance set forth in the following Table 2:

TABLE 2
APOC3 RNAi Drug Substance (ADS-005)
Sense and Antisense Strands
(The sense and antisense strands
are annealed to form a duplex):

Sense Strand (Modified Sequence)
(5′ → 3′):
(NAG37)s(invAb)sacgggacaGf
UfAfuucucaguias(invAb)
(SEQ ID NO: 6)
Antisense Strand (Modified Sequence)
(5′ → 3′):
usCfsasCfuGfagaauAfcUfgUfcCfcGfsu
(SEQ ID NO: 2)

TABLE 2.1
Properties of APOC3 RNAi Drug Substance
(ADS-005) Described in Table 2

| | |
|---|---|
| Chemical | C₄₉₃H₆₁₁F₁₁N₁₆₄Na₄₃O₃₁₁P₄₃S₇ (Na⁺ form) |
| Formula: | C₄₉₃H₆₅₄F₁₁N₁₆₄O₃₁₁P₄₃S₇ (H⁺ form) |
| Molecular | 16563.9 Da (Na+ form) |
| Weight: | 15618.7 Da (H+ form) |
| Physical | White to Off-white Powder |
| Appearance: | |

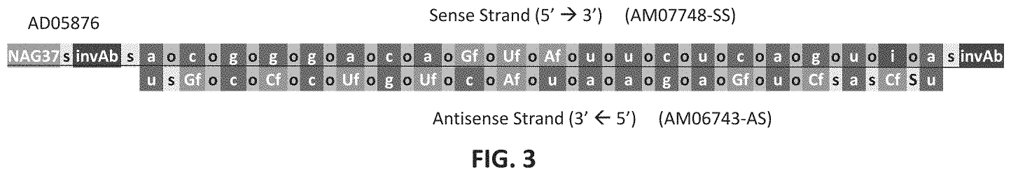

A schematic representation of APOC3 RNAi Drug Substance (ADS-005) is shown in FIG. 3, and full chemical structure representations are shown in FIGS. 1A to 1D (free acid form) and FIGS. 2A to 2D (sodium salt form). In some embodiments, the APOC3 RNAi Drug Substance is prepared or provided as a salt, mixed salt, or a free acid. In some embodiments, the form is a sodium salt.

### Pharmaceutical Compositions and Formulations

The APOC3 RNAi agents suitable for use in the methods disclosed herein can be prepared as pharmaceutical compositions or formulations for administration to human subjects. The pharmaceutical compositions can be used to treat a subject having a disease or disorder that would benefit from inhibition of expression of APOC3 mRNA or reduction in the level of APOC3 protein, such as human subjects having an APOC3-related disease or disorder. In some embodiments, the methods include administering an APOC3 RNAi agent that is linked to a targeting group or targeting ligand as described herein, to a subject in need of treatment. In some embodiments, one or more pharmaceutically acceptable excipients (including vehicles, carriers, diluents, and/or delivery polymers) are added to the pharmaceutical compositions that include an APOC3 RNAi agent, thereby forming a pharmaceutical formulation suitable for in vivo delivery to a human subject.

The pharmaceutical compositions that include an APOC3 RNAi agent, when administered to a human subject using the methods disclosed herein, decrease or reduce the level of APOC3 mRNA in the subject.

In some embodiments, the described pharmaceutical compositions including an APOC3 RNAi agent are used for treating or managing clinical presentations in a subject with an APOC3-related disease or disorder. In some embodiments, a therapeutically or prophylactically effective amount of one or more of pharmaceutical compositions is administered to a subject in need of such treatment. In some embodiments, administration of any of the disclosed APOC3 RNAi agents can be used to decrease the number, severity, and/or frequency of symptoms of a disease in a subject.

The described pharmaceutical compositions that include an APOC3 RNAi agent can be used to treat at least one symptom in a subject having a disease or disorder that would benefit from reduction or inhibition in expression of APOC3 protein levels. In some embodiments, the subject is administered a therapeutically effective amount of one or more pharmaceutical compositions including an APOC3 RNAi agent thereby treating the symptom. In other embodiments, the subject is administered a prophylactically effective amount of one or more APOC3 RNAi agents, thereby preventing the at least one symptom.

The APOC3 RNAi agents disclosed herein can be administered via any suitable route in a preparation appropriately tailored to the particular route. Thus, herein described pharmaceutical compositions can be administered by injection, for example, intravenously or subcutaneously. In some embodiments, the herein described pharmaceutical compositions are administered via subcutaneous injection.

As used herein, a pharmaceutical composition or medicament includes a pharmacologically effective amount of at least one APOC3 RNAi agents and one or more pharmaceutically acceptable excipients. Pharmaceutically acceptable excipients (excipients) are substances other than the Active Pharmaceutical Ingredient (API, therapeutic product, e.g., APOC3 RNAi agent) that are intentionally included in the drug delivery system. Excipients do not exert or are not intended to exert a therapeutic effect at the intended dosage. Excipients can act to a) aid in processing of the drug delivery system during manufacture, b) protect, support, or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients may include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble). For subcutaneous or intravenous administration, suitable carriers may include physiological saline, bacteriostatic water, Cremophor® EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above.

In some embodiments, a pharmaceutical composition suitable for use in the methods disclosed herein includes the components identified in the Formulated APOC3 RNAi Drug Substance provided in Table 3, below.

The APOC3 RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. In some embodiments, the dosage unit is between about 1 mg and about 100 mg of APOC3 RNAi Drug Substance. In some embodiments, the dosage unit is between about 10 mg and about 100 mg of APOC3 RNAi Drug Substance. In some embodiments, the dosage unit is between about 10 mg and about 50 mg of APOC3 RNAi Drug Substance. In some embodiments, the dosage unit is about 10 mg of APOC3 RNAi Drug Substance. In some embodiments, the dosage unit is about 25 mg of APOC3 RNAi Drug Substance. In some embodiments, the dosage unit is about 50 mg of APOC3 RNAi Drug Substance. In some embodiments, the dosage unit is about 100 mg of APOC3 RNAi Drug Substance. In some embodiments, the dosage unit is from about 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, or 98 to about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98 or 100 mg of APOC3 RNAi Drug Substance.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.).

As used herein, “pharmacologically effective amount,” “therapeutically effective amount,” or simply “effective amount” refers to that amount of an RNAi agent to produce a pharmacological, therapeutic or preventive result.

The described pharmaceutically acceptable formulations can be packaged into kits, containers, packs, or dispensers. The pharmaceutical compositions described herein can be packaged in pre-filled syringes or vials.

### Formulated APOC3 RNAi Drug Substance

In some embodiments, the APOC3 RNAi Drug Substance as provided in Table 2 (ADS-005) is formulated with one or more pharmaceutically acceptable excipients to form a pharmaceutical composition suitable for administration to a human subject. In some embodiments, the APOC3 RNAi Drug Substance described in Table 2 is formulated at 200 mg/mL in an aqueous sodium phosphate buffer (0.5 mM sodium phosphate monobasic, 0.5 mM sodium phosphate dibasic), forming the Formulated APOC3 RNAi Drug Substance (ADS-005-1) shown in Table 3:

TABLE 3
Composition of Formulated APOC3 RNAi
Drug Substance, per 1.0 mL

| | | Quality/ | Concen- |
|---|---|---|---|
| Component | Function | Grade | tration |
| ADS-005 | Active | In-house | 200 mg |
| | ingredient | | |
| Sodium phosphate | Suspending | USP, Ph. Eur | 0.062 mg |
| monobasic, | agent | | |
| monohydrate | | | |
| Sodium phosphate | Suspending | USP, Ph. Eur | 0.063 mg |
| dibasic, anhydrous | agent | | |
| Water for injection | Vehicle | USP, Ph. Eur | 890.6 mg |
| (WFI) | | | |

The Formulated APOC3 RNAi Drug Substance according to Table 3 is prepared as a sterile formulation. In some embodiments, the Formulated APOC3 RNAi Drug Substance is packaged in a container, such as a glass vial. In some embodiments, the Formulated APOC3 RNAi Drug Substance is packaged in a glass vial with a fill volume of about 1.2 mL, and a desired volume for administration can be calculated based upon the desired dose level to be administered.

In some embodiments, the Formulated APOC3 RNAi Drug Substance set forth in Table 3 is administered to a human subject using the methods disclosed herein.

Human Subjects with Elevated Triglyceride Levels and/or Over-Expression of APOC3

The methods disclosed herein include treating diseases, disorders, or other conditions or symptoms that can be ameliorated at least in part by a reduction in or the silencing of APOC3 gene expression in a human subject in need thereof, using pharmaceutical compositions that include the APOC3 RNAi Drug Substance described in Table 2. In some embodiments, the methods disclosed herein include treating diseases or conditions associated with elevated triglycerides in a human subject in need thereof, using pharmaceutical compositions that include the APOC3 RNAi Drug Substance described in Table 2. In some embodiments, prior to administration the human subject is diagnosed with an APOC3-related disease or disorder. In some embodiments, the human subject has been diagnosed as having cardiovascular disease, coronary artery disease, or atherosclerosis. In some embodiments, the human subject has been diagnosed as having moderate hypertriglyceridemia (TG levels of 150-499 mg/dL according to the 2018 AHA/ACC Guidelines), severe hypertriglyceridemia (TG >500 mg/dL), and/or chylomicronemia and/or familial chylomicronemia syndrome (FCS) (TG levels often >1000 mg/dL). FCS is a severe rare genetic disease, with a prevalence of 1 in 1,000,000, often caused by various monogenic mutations (e.g. null mutations in LPL, GPIHBP1, APOC2, APOA5 or LMF1) leading to extremely high TG levels, typically over 900 mg/dL. As shown in the Examples herein, the APOC3 RNAi Drug Substance described in Table 2 provides for a substantial TG lowering effect suitable for the treatment of diseases and disorders.

### Dosing and Inhibition of APOC3 Gene Expression

Generally, an effective amount of an APOC3 RNAi agent will be in the range of from about 0.1 to about 10 mg/kg of body weight per dose, e.g., from about 0.25 to about 5 mg/kg of body weight per dose. In some embodiments, an effective amount of an APOC3 RNAi agent will be in the range of from about 0.5 to about 4 mg/kg of body weight per dose. In some embodiments, the effective amount is a fixed dose. In some embodiments, a fixed dose of between 1 mg to 100 mg of APOC3 RNAi Drug Substance is an effective dose. In some embodiments, a fixed dose of between 10 mg to 50 mg of APOC3 RNAi Drug Substance is an effective dose. In some embodiments, a fixed dose of between 20 mg to 50 mg of APOC3 RNAi Drug Substance is an effective dose. In some embodiments, a fixed dose of between 25 mg to 50 mg of APOC3 RNAi Drug Substance is an effective dose. The amount administered will likely depend on such variables as the overall age and health status of the patient, the relative biological efficacy of the compound delivered, the formulation of the drug, the presence and types of excipients in the formulation, and the route of administration. In some embodiments, a fixed dose of from about 5, 10, 15, 20, 25, 30, 35, 40, 45, 50, 55, 60, 65, 70, 75, 80, 85, 90, 95, or 100 mg is an effective dose. In some embodiments, a fixed dose of about 10 mg, about 25 mg, about 50 mg, or about 100 mg is an effective dose.

Also, it is to be understood that the initial dosage administered can, in some instances, be increased beyond the above upper level to rapidly achieve the desired blood-level or tissue level, or the initial dosage can, in some instances, be smaller than the optimum. For example, in some embodiments, an initial dose or a first dose of from about 1 mg to about 100 mg of APOC3 RNAi Drug Substance is administered, followed by a second dose of from about 1 to 100 mg of APOC3 RNAi Drug Substance approximately 1 month later, and thereafter additional doses (a concept similar to “maintenance doses”) are administered once every three months (e.g., once per calendar quarter or once per 12 weeks (q12w)).

For treatment of disease or for formation of a medicament or composition for treatment of a disease, the pharmaceutical compositions described herein including an APOC3 RNAi agent can be combined with an excipient or with a second therapeutic agent or treatment including, but not limited to: a second or other RNAi agent, a small molecule drug, an antibody, an antibody fragment, peptide and/or aptamer.

In some embodiments, the APOC3 mRNA level of a subject to whom a described APOC3 RNAi agent is administered is reduced in the liver by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%. In some embodiments, the APOC3 mRNA level of a subject to whom a described APOC3 RNAi agent is administered is reduced in the entire subject by at least about 5%, 10%, 15%, 20%, 25%, 30%, 35%, 40%, 45%, 50%, 55%, 60%, 65%, 70%, 75%, 80%, or greater than 80% relative to the subject prior to being administered the APOC3 RNAi agent or to a subject not receiving the APOC3 RNAi agent. The gene expression level and/or mRNA level in the subject is reduced in a cell, group of cells, and/or tissue of the subject.

In some embodiments, the APOC3 protein levels in a subject to whom a described APOC3 RNAi agent has been administered is reduced in the liver by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the APOC3 RNAi agent or to a subject not receiving the APOC3 RNAi agent. In some embodiments, the APOC3 protein levels in a subject to whom a described APOC3 RNAi agent has been administered is reduced in the entire subject by at least about 5%, 10%, 15%, 20%, 25%, 30%, 35%, 40%, 45%, 50%, 55%, 60%, 65%, 70%, 75%, 80%, or greater than 80% relative to the subject prior to being administered the APOC3 RNAi agent or to a subject not receiving the APOC3 RNAi agent. The protein level in the subject can be reduced in a cell, group of cells, tissue, blood, and/or other fluid of the subject.

A reduction in APOC3 gene expression, APOC3 mRNA, or APOC3 protein levels can be assessed and quantified by general methods known in the art. The Examples disclosed herein forth generally known methods for assessing inhibition of APOC3 gene expression and reduction in APOC3 protein levels. The reduction or decrease in APOC3 mRNA level and/or protein level are collectively referred to herein as a reduction or decrease in APOC3 or inhibiting or reducing the expression of APOC3.

In some embodiments, the triglyceride level in a subject to whom a described APOC3 RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, or greater than 95% relative to the subject prior to being administered the APOC3 RNAi agent or to a subject not receiving the APOC3 RNAi agent. The triglyceride level in the subject is reduced in the blood/serum of the subject.

In some embodiments, the very low density lipoprotein cholesterol (VLDL-C) levels in a subject to whom a described APOC3 RNAi agent has been administered is reduced by at least about 5%, 10%, 15%, 20%, 25%, 30%, 35%, 40%, 45%, 50%, 55%, 60%, 65%, 70%, 75%, 80%, 85%, or greater than 85% relative to the subject prior to being administered the APOC3 RNAi agent or to a subject not receiving the APOC3 RNAi agent. The VLDL-C level in the subject is reduced in the blood/serum of the subject.

In some embodiments, the total cholesterol levels in a subject to whom a described APOC3 RNAi agent has been administered is reduced by at least about 5%, 10%, 15%, 20%, 25%, or greater than 25% relative to the subject prior to being administered the APOC3 RNAi agent or to a subject not receiving the APOC3 RNAi agent. The total cholesterol levels in the subject is reduced in a cell, group of cells, tissue, blood, and/or other fluid of the subject.

In some embodiments, the high-density lipoprotein cholesterol levels (HDL-C) in a subject to whom a described APOC3 RNAi agent has been administered is increased by at least about 5%, 10%, 15%, 20%, 25%, 30%, 35%, 40%, 45%, 50%, 55%, 60%, 65%, 70%, 75%, 80%, 85%, or greater than 85% relative to the subject prior to being administered the APOC3 RNAi agent or to a subject not receiving the APOC3 RNAi agent. The total HDL-C levels in the subject is increased in a cell, group of cells, tissue, blood, and/or other fluid of the subject.

In some embodiments, the apolipoprotein A1 (ApoA1) levels in a subject to whom a described APOC3 RNAi agent has been administered is increased by at least about 5%, 10%, 15%, 20%, 25%, 30%, 35%, 40%, 45%, 50%, 55%, 60%, 65%, 70%, 75%, 80%, 85%, 90%, 95%, or greater than 95% relative to the subject prior to being administered the APOC3 RNAi agent or to a subject not receiving the APOC3 RNAi agent. The ApoA1 levels in the subject is reduced in a cell, group of cells, tissue, blood, and/or other fluid of the subject.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition of the number, severity, and/or frequency of one or more symptoms of a disease in a subject. As used herein, unless otherwise expressly noted, the phrase “treatment of a disease or disorder” includes the treatment of underlying conditions and/or symptoms of the disease, as would be understood by the person of ordinary skill in the art. Unless otherwise expressly limited herein, such phrases are not intended to be limiting.

As used herein, an “APOC3-related disease or disorder” refers to any disease or disorder that can be treated at least in part by a reduction or inhibition in APOC3 gene expression using the APOC3 RNAi agent, including by the methods of treatment described herein. As discussed herein, the methods of treatment disclosed herein, among other things, result in a reduction in TG levels in a subject to which the APOC3 RNAi Drug Substance is administered. APOC3-related diseases and disorders include, but are not limited to, familial chylomicronemia syndrome (FCS), chylomicronemia, multifactorial chylomicronemia, hypertriglyceridemia induced pancreatitis, metabolic syndrome, type II diabetes mellitus, lipodystrophy syndromes including familial partial lipodystrophy, obesity, dyslipidemia, non-alcoholic steatohepatitis, non-alcoholic fatty liver disease, hyperlipidemia, hypertriglyceridemia, abnormal lipid and/or cholesterol metabolism, atherosclerosis, cardiovascular disease, coronary artery disease, and other dyslipidemias and metabolic-related disorders and diseases.

As used herein, “monthly dosing” or “monthly” administration means every 28 days. As used herein, “quarterly dosing” or “quarterly” administration means every 84 days. The term “about” when used in connection with monthly dosing means monthly dosing+/−5 days. The term “about” when used in connection with quarterly dosing means quarterly dosing+/−14 days.

As used herein, the phrase “introducing into a cell,” when referring to an RNAi agent, means functionally delivering the RNAi agent into a cell. The phrase “functional delivery,” means that delivering the RNAi agent to the cell in a manner that enables the RNAi agent to have the expected biological activity, e.g., sequence-specific inhibition of gene expression.

Unless stated otherwise, use of the symbol

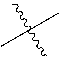

as used herein means that any group or groups may be linked thereto that is in accordance with the scope of the inventions described herein.

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The above provided embodiments and items are now illustrated with the following, non-limiting examples.

### EXAMPLES

### Example 1. Synthesis and Formulation of APOC3 RNAi Drug Substance (ADS-005)

The APOC3 RNAi Drug Substance suitable for use in the methods disclosed herein can be synthesized using standard phosphoramidite technology on solid phase oligonucleotide synthesis as is known in the art. Commercially available oligonucleotide synthesizers (e.g., MerMade96E® (Bioautomation) or MerMade12® (Bioautomation)) may be used. Syntheses can be performed on a solid support made of controlled pore glass (CPG, 500 Å or 600 Å, obtained from Prime Synthesis, Aston, Pa., USA). The monomer positioned at the 3′ end of the respective strand may be attached to the solid support as a starting point for synthesis. All RNA, 2′-modified RNA phosphoramidites, and inverted abasic phosphoramidites can be purchased commercially. Targeting group-containing phosphoramidites can be synthesized that are suitable for addition to the 5′ end of the sense strand. Standard cleavage, deprotection, purification, and annealing steps can be utilized as is known in the art. Further description related to the synthesis of APOC3 RNAi agents may be found, for example, in International Patent Application Publication No. WO 2019/051402 (Patent Application No. PCT/US2018/050248) and WO 2018/044350 (PCT/US2017/021147), each of which is incorporated by reference herein in its entirety. APOC3 RNAi Drug Substance can then be formulated by dissolving in standard pharmaceutically acceptable excipients that are generally known in the art. For example, Table 3 shows a Formulated APOC3 RNAi Drug Substance that is suitable for use in the methods disclosed herein.

### Example 2. Phase I Clinical Trial of APOC3 RNAi Drug Substance (ADS-005) in Healthy Human Volunteers and Severely Hypertriglyceridemic Patients and Patients with Familial Chylomicronemia Syndrome (FCS)

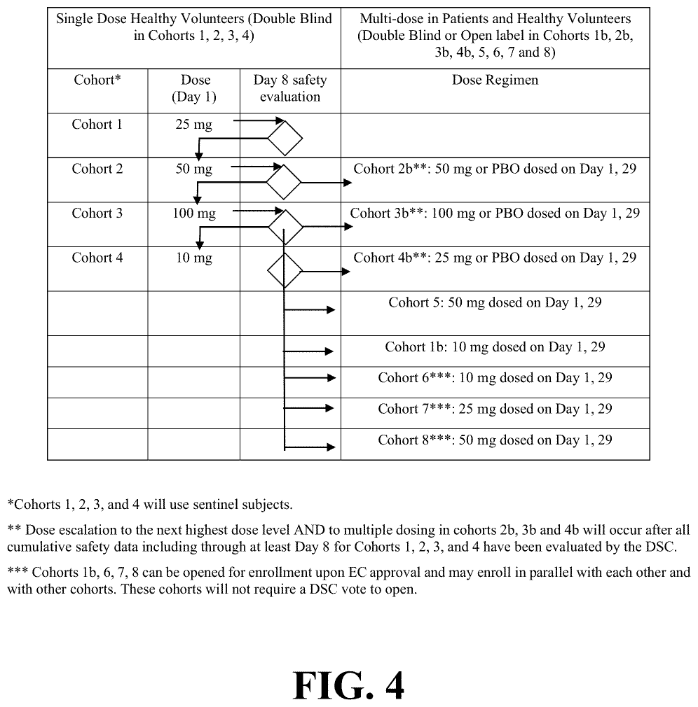

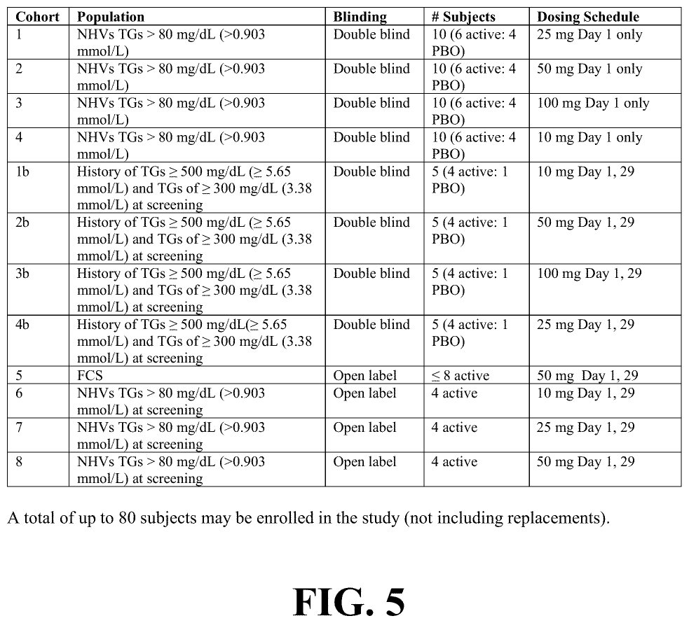

A Phase 1, single and multiple dose-escalating study to evaluate the safety, tolerability, pharmacokinetics and pharmacodynamic effects of APOC3 RNAi Drug Substance (ADS-005) in adult healthy volunteers as well as in severely hypertriglyceridemic patients and patients with FCS was undertaken. The study subject population includes adult males and females 18-65 years old with a BMI between 19.0 and 40.0 kg/m², and:
Cohorts 1, 2, 3 and 4: All subjects have fasting screening triglycerides >80 mg/dL (>0.903 mmol/L) and are not on any lipid or triglyceride lowering therapy. Each double-blind cohort will enroll ten (10) subjects (6 active: 4 placebo (PBO)) with all cohorts to receive single escalating doses of APOC3 RNAi Drug Substance or PBO at escalating dose levels of 10, 25, 50, and 100 mg as per FIGS. 4 and 5.
Cohort 1b, 2b, 3b and 4b: Cohorts are double blind with 8 active and 2 placebo patients per cohort with fasting serum triglycerides of at least 300 mg/dL (3.38 mmol/L) at screening. All subjects to receive multiple escalating doses of APOC3 RNAi Drug Substance or placebo at dose levels of 10, 25, 50 or 100 mg.
Cohorts 5: Cohort is open label with up to 8 chylomicronemia patients. Cohort 5 subjects will each receive two 50 mg doses of APOC3 RNAi Drug Substance.
Cohorts 6, 7, and 8: All subjects have fasting screening triglycerides >80 mg/dL (>0.903 mmol/L) and are not on any lipid or triglyceride lowering therapy. Each double-blind cohort will enroll ten (10) subjects (6 active: 4 placebo (PBO)) with all cohorts to receive single escalating doses of APOC3 RNAi Drug Substance or PBO at escalating dose levels of 10, 25, 50, and 100 mg as per FIGS. 4 and 5.

Subjects will be separated into various cohorts in view of the criteria discussed above. (See also FIG. 5) Cohorts 1 through 4 were randomized to receive APOC3 RNAi Drug Substance or placebo (6 active: 4 placebo) at single doses of 25 mg (Cohort 1), 50 mg (Cohort 2), 100 mg (Cohort 3), or 10 mg (Cohort 4), administered as a subcutaneous injection. In other cohorts, two doses (administered on days 1 and 29) of 10 mg (Cohort 1b and Cohort 6), 50 mg (Cohort 2b and Cohort 8), 100 mg (Cohort 3b), or 25 mg (Cohort 4b and Cohort 7) will be administered via subcutaneous injections. In addition, two doses of 50 mg will be administered to chylomicronemia patients (Cohort 5). Cohorts 1, 2, 3, 4, 1b, 2b, 3b, and 4b are double-blinded. Cohorts 5, 6, 7 and 8 are open label. A total of up to 80 subjects may be enrolled in the study. FIG. 4 shows the study design for the Phase I Clinical Trial, as amended. The study parameters are summarized in the following Table 4.

TABLE 4
Phase I Clinical Study Parameters

| Develop- | |
|---|---|
| ment | |
| Phase | Phase 1: First-in-Human |
| Study | Primary Objectives: |
| Objectives | To determine the incidence and frequency |
| | of adverse events possibly or probably |
| | related to treatment as a measure of the |
| | safety and tolerability of APOC3 RNAi |
| | Drug Substance (ADS-005) using escalating |
| | single doses in healthy volunteers and escalating |
| | multiple doses in severely hypertriglyceridemic |
| | patients and patients with familial |
| | chylomicronemia syndrome (FCS). |
| | Secondary Objectives: |
| | To evaluate the single-dose pharmacokinetics |
| | of APOC3 RNAi Drug Substance in healthy |
| | volunteers. |
| | To determine the reduction in fasting serum |
| | APOC3 from baseline in response to single |
| | doses of APOC3 RNAi Drug Substance as a |
| | measure of drug activity in healthy volunteers |
| | and multiple doses in severely hypertriglyceridemia |
| | patients and patients with FCS. |
| | Exploratory Objectives: |
| | To evaluate the effect of APOC3 RNAi Drug |
| | Substance on change from baseline in fasting |
| | LDL-C, Total Cholesterol, non-HDL-C, HDL-C, |
| | VLDL-C, Triglycerides, apoB-48, apoB-100, |
| | APOC3, apoC-II, apoA-I, lipoprotein lipase |
| | mass (if feasible), hepatic lipase mass (if feasible), |
| | CETP mass (if feasible) and apoA-V (all values |
| | drawn after at least 8-hour fast). |
| | To evaluate the effect of APOC3 RNAi Drug |
| | Substance on changes from baseline in BMI. |
| | To evaluate the effect of APOC3 RNAi Drug |
| | Substance on changes from baseline in fasting |
| | serum blood glucose, hemoglobin A1C, C-peptide, |
| | GTT and fasting serum insulin. |
| | To evaluate the effect of APOC3 RNAi Drug |
| | Substance on change from baseline in post-prandial |
| | (post standardized high fat/high carbohydrate meal) |
| | serum TGs in specified cohorts. |
| | To evaluate excretion of ARO-APOC3 (full length |
| | and metabolites) and identify metabolites in plasma |
| | and urine in the multi-dose healthy volunteer cohorts. |
| Study | Cohorts 1 through 4: randomized, double-blind, |
| Design | placebo-controlled |
| | Cohorts 1b, 2b, 3b, and 4b: randomized, double-blind, |
| | placebo-controlled |
| | Cohort 5, 6, 7, and 8: open label |
| Study | This study was conducted in healthy volunteers, |
| Population | adult males and females, aged 18-65 years with BMI |
| | between 19.0 and 40.0 kg/m². |
| Investi- | APOC3 RNAi Drug Substance (ADS-005) (see Table |
| gational | 2), administered as Formulated APOC3 RNAi Drug |
| Product | Substance (see Table 3) |
| Dosage and | Cohorts 1-4: randomized to receive APOC3 RNAi |
| Frequency | Drug Substance (ADS-005) or placebo (6 active: |
| | 4 placebo) at a single dose of 25 mg, 50 mg, 100 mg, |
| | or 10 mg, administered as a single subcutaneous injection. |
| | Cohorts 1b, 2b, 3b, and 4b: will be randomized to receive |
| | two monthly (i.e., days 1 and 29) doses of 10 mg (Cohort |
| | 1b), 50 mg (Cohort 2b), 100 mg (Cohort 3b), or 25 mg |
| | (Cohort 4b) of APOC3 RNAi Drug Substance or placebo |
| | (4 active: 1 placebo), via subcutaneous injection. |
| | Cohort 5: will be enrolled to receive two monthly (i.e., |
| | days 1 and 29) doses of 50 mg of APOC3 RNAi Drug |
| | Substance (all 8 active) via subcutaneous injection. |
| | Cohort 6-8: Open label, will be enrolled to receive two |
| | monthly doses of either 10 mg, 25 mg or 50 mg of |
| | APOC3 RNAi Drug Substance (ADS-005), |
| | administered by subcutaneous injection. |
| Reference | Placebo (PBO): normal saline (0.9%) administered |
| Formulation | subcutaneously with matching volume. |
| Safety | Safety will be assessed by adverse events, serious adverse |
| Evaluation | events, physical examinations, vital sign measurements |
| Criteria | (blood pressure, heart rate, temperature, and respiratory |
| | rate), ECG measurements, clinical laboratory tests, |
| | concomitant medications/therapy, and reasons for |
| | treatment discontinuation. and 90-day post-last dose |
| | pregnancy follow up phone call. |
| Pharmaco- | Blood samples will be collected from each subject for |
| kinetics | pharmacokinetic analysis after dose 1 (Cohorts 1, 2, |
| Evaluation | 3, and 4) per a schedule of assessments. Blood and |
| | Urine samples will be collected from each subject after |
| | both doses (Cohorts 6, 7, 8) per a schedule of |
| | assessments. |
| Data | Screening, Compliance, Tolerability and Safety Data: |
| Analysis | Safety analyses will be performed, and the results |
| | summarized by cohort. The incidence and frequency |
| | of adverse events (AEs), serious adverse events (SAEs), |
| | related AEs, related SAEs, and AEs leading to |
| | discontinuation, will be summarized by cohort per |
| | SOC, PT, and severity. Other safety parameters will |
| | be summarized at each scheduled time. |
| | Pharmacodynamic analysis: |
| | Data will be summarized by cohort as applicable for |
| | the following: |
| | Serum APOC3, apoC-II, LDL-C, Total Cholesterol, |
| | non-HDL-C, HDL-C, VLDL-C, Triglycerides, apoB-48, |
| | apoB-100, apoA-I, apoA-V, hemoglobin A1C, C-peptide, |
| | changes in serum insulin, changes in serum glucose, |
| | changes in GTT, changes in CETP mass, lipoprotein |
| | lipase mass and hepatic lipase mass. For lipid related, |
| | lipoprotein and serum pharmacodynamic assessments, |
| | baseline is defined as the pre-dose value obtained |
| | nearest to the first dose. |
| | Percent change from Day 1 pre-dose baseline to nadir |
| | for each serum marker |
| | Duration of response from nadir back to above 50% of |
| | baseline for each serum marker (if available by EOS) |
| | Descriptive statistics of biomarker (SerumAPOC3, |
| | apoC-II, LDL-C, Total Cholesterol, non-HDL-C, |
| | HDL-C, VLDL-C, Triglycerides, apoB-100, apoB-48, |
| | apoA-I, apoA-V, lipoprotein lipase mass (if feasible), |
| | hepatic lipase mass (if feasible), CETP mass (if |
| | feasible), Hemoglobin A1C, C-peptide, serum glucose, |
| | serum insulin, GTT,) changes will include mean, median |
| | SD, minimum, and maximum. Additional details will be |
| | provided in the statistical analysis plan. |
| | Pre-specified separate analysis will be performed for |
| | subjects by cohort, for subjects with elevated baseline |
| | Hemoglobin A1C and/or diabetes mellitus. Separate |
| | analyses will be completed for all subjects with history |
| | of TGs ≥ 500 mg/dL (≥5.65 mmol/L) together (combined |
| | for cohorts 2b, 3b and 4b) and for cohorts 2b, 3b, 4b and |
| | 5 combined, in additional to analysis performed for each |
| | cohort. |
| | Pharmacokinetics: |
| | Plasma concentrations of APOC3 RNAi Drug Substance |
| | product constituents will be used to calculate the following |
| | PK parameters: |
| | maximum observed plasma concentration (Cₘₐₓ), area under |
| | the plasma concentration time curve (AUC) from time 0 to |
| | 24 hours (AUC₀₋₂₄), AUC from time 0 extrapolated to |
| | infinity (AUC_{inf}), and terminal elimination half-life (t_{1/2}). |
| | Pharmacokinetic parameters will be determined using non- |
| | compartmental methods. Descriptive statistics of PK |
| | parameters will include mean, standard deviation |
| | (SD), coefficient of variation, median, minimum, and |
| | maximum. PK results will be analyzed for dose |
| | proportionality, and sex differences. |
| | Excretion and Metabolism: |
| | Urine collections will be performed (Cohorts 6, 7, 8) for |
| | excretion and metabolic analysis after dose 1 and dose 2 |
| | (Day 29) per the Schedule of Assessments, along with spot |
| | checks between doses to measure elimination PK. |
| | PK population: All healthy volunteer (Cohorts 1, 2, 3 4, 6, |
| | 7 and 8) subjects that received at least one dose of active |
| | study treatment (APOC3 RNAi Drug Substance). |
| | Excretion and metabolism population: All healthy |
| | volunteer cohorts (cohorts 6, 7, and 8) that received two |
| | doses of active study treatment (APOC3 RNAi Drug |
| | Substance). |

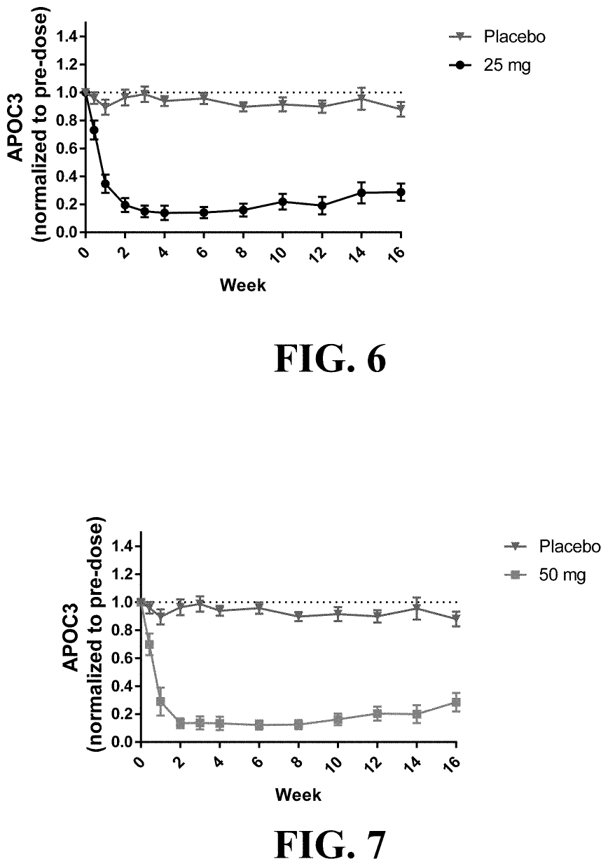

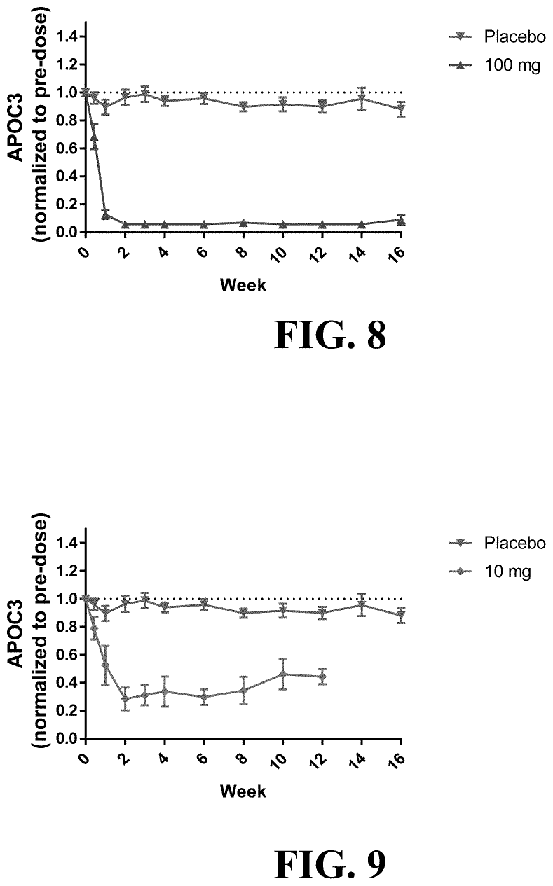

Initially, a cohort was proposed as part of the clinical trial protocol at 200 mg of APOC3 RNAi Drug Substance per dose. However, in view of the unexpected and surprising potency at the 25 and 50 mg doses, the study protocol was amended to remove the 200 mg cohort. The 200 mg cohort was replaced with a cohort dosed at a lower dose of 10 mg. Doses of 10 mg, 25 mg, 50 mg, and 100 mg all yielded substantial serum APOC3 reductions. For example, both the 25 mg and 50 mg cohorts reached approximately 85% mean serum APOC3 protein reduction after a single dose in the Phase I study by day 22, and the 100 mg cohort reached approximately 85% mean serum APOC3 protein reduction after a single dose in the Phase I study by day 8. FIGS. 6 through 9 report on the serum APOC3 reductions of Cohorts 1, 2, 3, and 4 in the Phase I study.

Serum APOC3 reduction results from the study show that administration of APOC3 RNAi Drug Substance, at least from doses of 10 mg, 25 mg, 50 mg, and 100 mg, resulted in deep and durable reduction of serum APOC3 when compared with placebo. As noted herein, while APOC3 is primarily synthesized in hepatocytes in the liver, data suggest that as much as 20% of APOC3 protein may be expressed in enterocytes in the gastrointestinal tract. Surprisingly and unexpectedly, the dose level at only 25 mg of APOC3 RNAi Drug Substance produced substantial knockdown of APOC3 gene expression reaching approximately 85% (which was similar knockdown levels observed with the higher 50 mg and 100 mg doses), indicating achievement of near-total suppression of hepatocyte-expressed APOC3. Also unexpectedly, the dose level at only 10 mg of APOC3 RNAi Drug Substance produced substantial knockdown, showing a reduction of up to approximately 72% of APOC3 gene expression compared to baseline values.

Duration of serum APOC3 reduction (>80%) from a single-dose of 25 mg appears unexpectedly durable as well. Through 85 days post dose administration, APOC3 reductions of ≥70% were observed at Day 113 (end of study). At the 10 mg dose, duration of effect was also long, with APOC3 reductions >60% observed through 57 days post-dose administration. These data show that monthly, quarterly, or even dosing once every 6 months at 10 mg, 25 mg, 50 mg, or 100 mg can achieve substantial APOC3 gene expression inhibition.

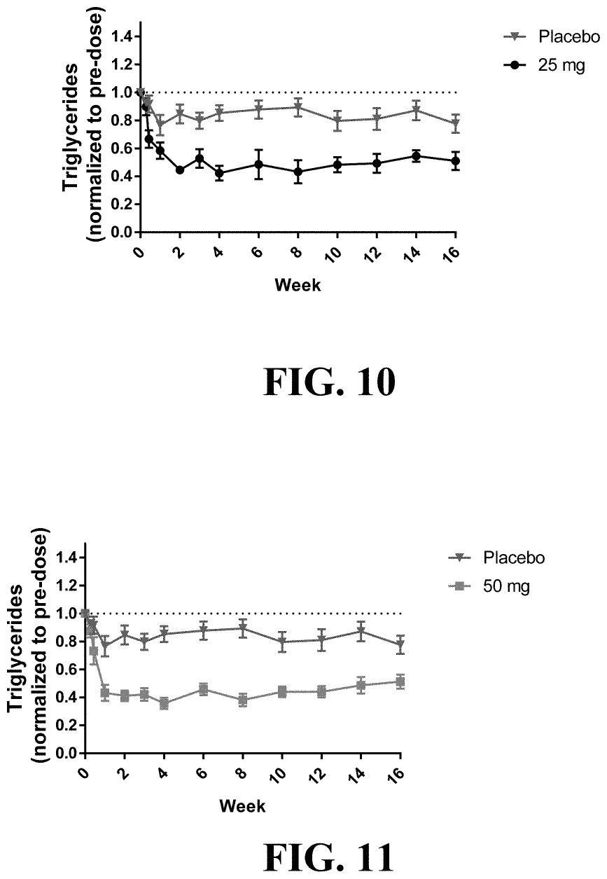

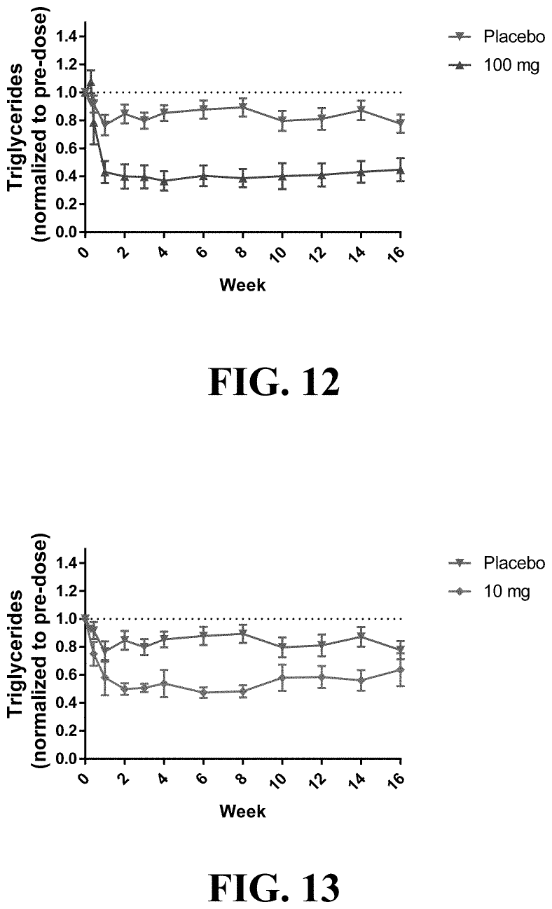

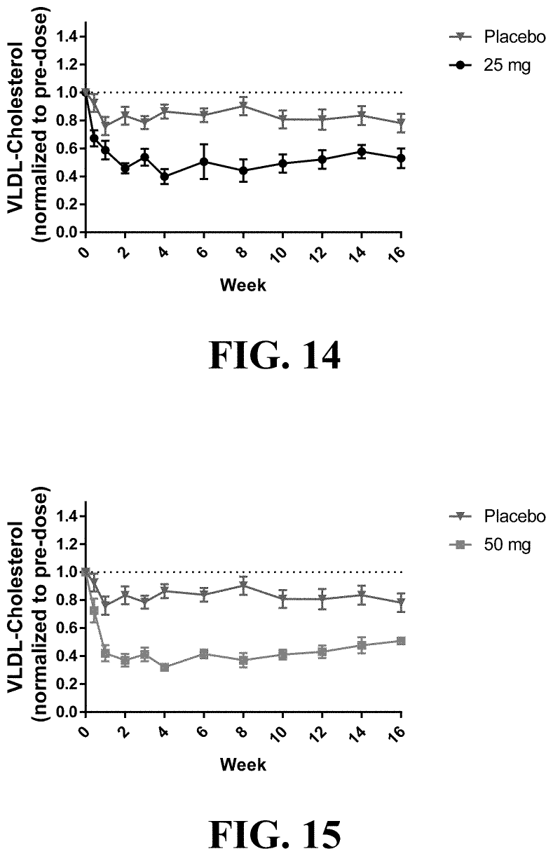

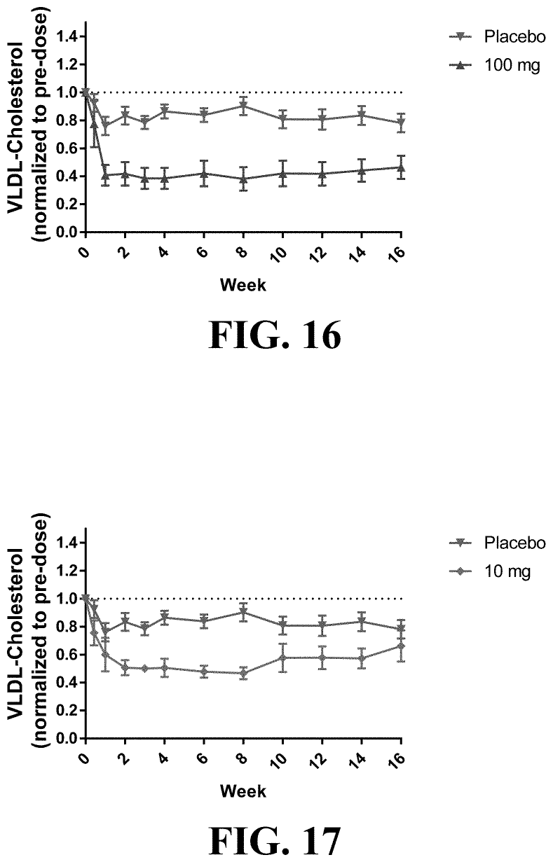

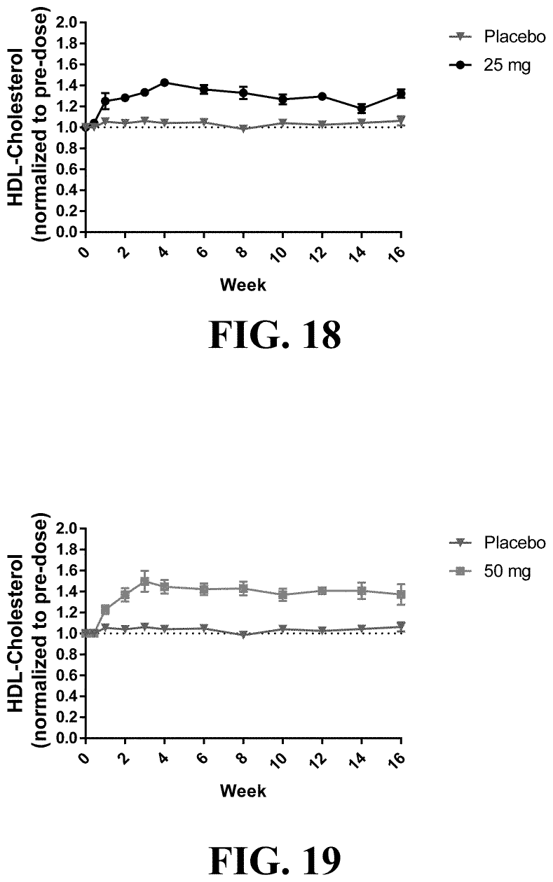

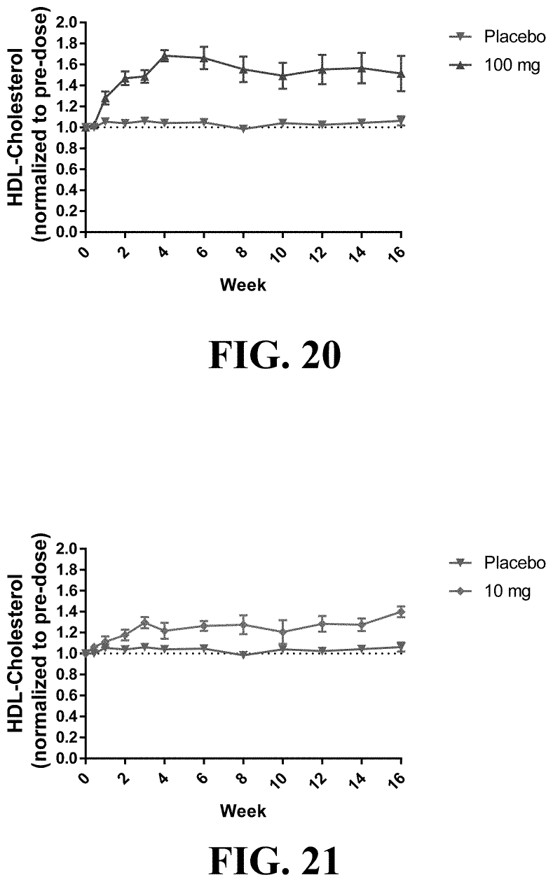

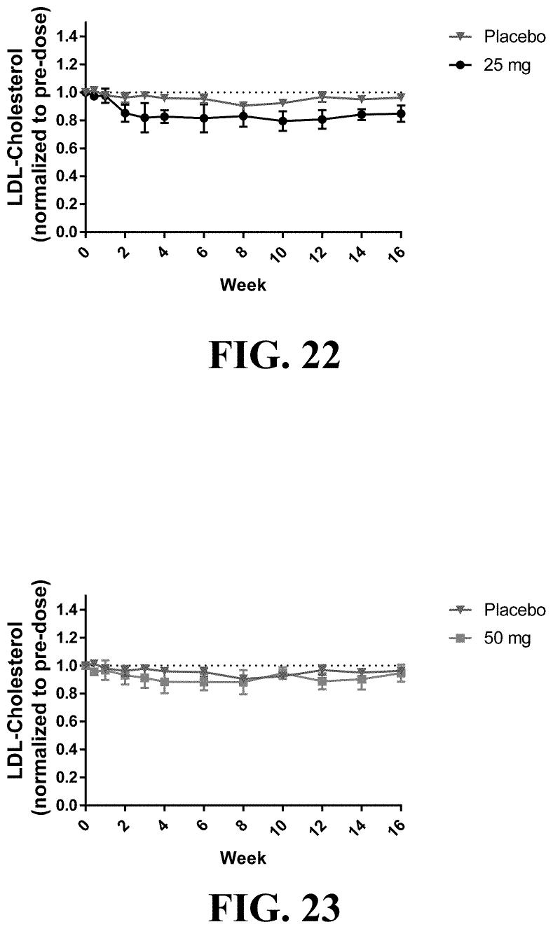

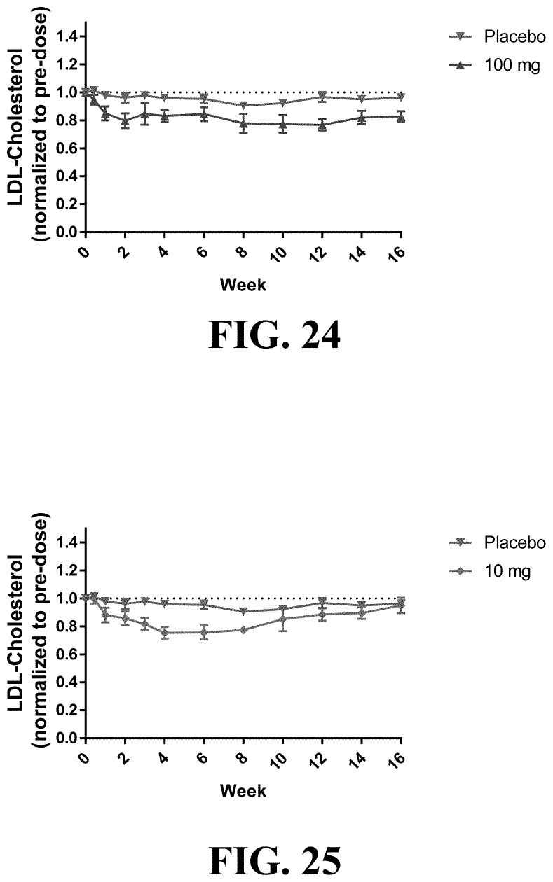

Substantial reductions in serum triglyceride (TG) levels were also observed across the various cohorts after administration of only a single dose. FIGS. 9 through 12 report on the serum TG reductions of Cohorts 1, 2, 3, and 4 in the Phase I study. Each of the 25 mg (Cohort 1), 50 mg (Cohort 2), and 100 mg (Cohort 3) dosing groups reached a maximal TG reduction of approximately 60% compared to baseline. Through day 113 (end of study), both the 25 mg (Cohort 1) and 50 mg (Cohort 2) maintained reductions in serum TGs of approximately 50%. Further, the 10 mg (Cohort 4) dosing group achieved a maximal TG reduction of approximately 50%, and maintained a reduction in serum TG of approximately 40% at day 99.

Similar reductions in VLDL-C to those of serum TGs we also observed. For example, on Day 29, the 25 mg (Cohort 1), 50 mg (Cohort 2), and 100 mg (Cohort 3) dosing groups each achieved approximately 60% reductions in VLDL-C levels, and at day 29, the 10 mg dosing group (Cohort 4) reported a reduction of approximately 50%. Further, the data show reductions of approximately 10% to 20% of total cholesterol and low density lipoprotein cholesterol (LDL-C) in subjects.

Additionally, administration of the APOC3 RNAi Drug Substance at the dose levels disclosed herein has shown increases of approximately 50% to 70% in high density lipoprotein cholesterol (HDL-C) and apolipoprotein A1 (APOA1) levels in the Phase 1 study. It has been observed from the Phase 1 study that HDL-C increases are slightly greater than those seen in APOC3-deficient individuals (homozygous or heterozygous).

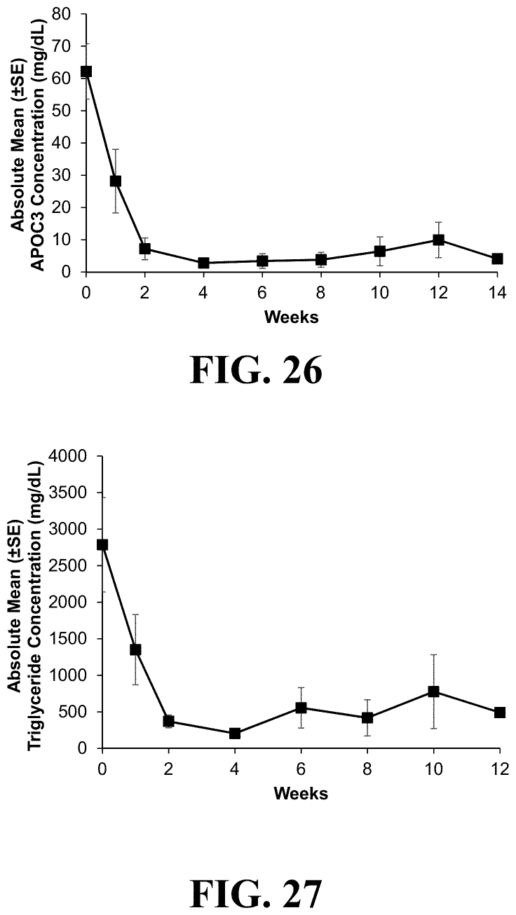

Multiple-dose data from patients in Cohort 5 (n=5, all chylomicronemia patients with data through day 29), also showed reductions in serum APOC3 protein levels and lipid parameters. With respect to APOC3 protein levels, maximal mean reductions of 96% (−62 mg/dL) were observed across three subjects, with mean baseline APOC3 levels of 66 mg/dL. In these five subjects, maximal mean reduction in triglycerides of 92% (−2583 mg/dL) with a mean baseline value of 2787 mg/dL±1444 mg/dL (standard deviation) were observed. In one of the subjects, maximal individual reduction of 99% (−86.7 mg/dL) was observed after a single dose with a starting concentration of 88 mg/dL. Further, a maximum individual reduction of 95% (−4410 mg/dL) was observed in one subject 28 days after the first dose where the subject had a starting triglyceride value of 4636 mg/dL. FIG. 26 shows APOC3 protein levels and FIG. 27 shows triglyceride levels from Cohort 5.

To date in the Phase I study there have been no deaths, no serious adverse events (SAEs), and no adverse events (AEs) rated as severe in intensity. Most AEs have been reported as mild.

### Other Embodiments

In some embodiments, disclosed herein are methods for treating an APOC3-related disease or disorder in a human subject in need thereof, the method comprising administering to the subject a pharmaceutical composition that comprises an APOC3 RNAi agent at a first dose of between about 1 mg to about 100 mg of the APOC3 RNAi agent, and administering to the subject a second dose of the pharmaceutical composition comprising between about 1 mg to about 100 mg of the APOC3 RNAi agent about one month after the first dose, wherein the first dose and the second dose are administered by subcutaneous injection.

In some embodiments, disclosed herein are methods for treating an APOC3-related disease or disorder in a human subject in need thereof, the method comprising administering to the subject a pharmaceutical composition that comprises an APOC3 RNAi agent at a first dose of between about 1 mg to about 100 mg of the APOC3 RNAi agent, and administering to the subject a second dose of the pharmaceutical composition comprising between about 1 mg to about 100 mg of the APOC3 RNAi agent about three months (e.g., quarterly dosing or dosing every 12 weeks (q12w)) after the first dose, wherein the first dose and the second dose are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi agent at a first dose of between about 1 mg to about 100 mg of the APOC3 RNAi agent, and administering to the subject a second dose of the pharmaceutical composition comprising between about 1 mg to about 100 mg of the APOC3 RNAi agent about four months after the first dose, wherein the first dose and the second dose are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi agent at a first dose of between about 1 mg to about 100 mg of the APOC3 RNAi agent, and administering to the subject a second dose of the pharmaceutical composition comprising between about 1 mg to about 100 mg of the APOC3 RNAi agent about six months after the first dose, wherein the first dose and the second dose are administered by subcutaneous injection.

In some embodiments, disclosed herein are methods for treating an APOC3-related disease or disorder in a human subject in need thereof, the method comprising:
a. administering to the human subject by subcutaneous administration a first dose of a pharmaceutical composition that comprises the APOC3 RNAi agent at a dose of between about 1 mg to about 100 mg of the APOC3 RNAi Drug Substance,
b. administering to the human subject a second dose of the pharmaceutical composition about one month after the first dose, and
c. administering to the human subject a third dose of the pharmaceutical composition about three to about six months after the second dose.

In some embodiments, disclosed herein are methods for treating an APOC3-related disease or disorder in a human subject in need thereof, the method comprising:
a. administering to the human subject by subcutaneous administration a first dose of a pharmaceutical composition that comprises the APOC3 RNAi agent at a dose of between about 1 mg to about 100 mg of the APOC3 RNAi agent,
b. administering to the human subject a second dose of the pharmaceutical composition about one month after the first dose, and
c. administering to the human subject a third dose of the pharmaceutical composition about six months after the second dose.

In some embodiments, disclosed herein are methods for treating an APOC3-related disease or disorder in a human subject in need thereof, the method comprising:
a. administering to the human subject by subcutaneous administration a first dose of a pharmaceutical composition that comprises the APOC3 RNAi agent at a dose of between about 1 mg to about 100 mg of the APOC3 RNAi agent,
b. administering to the human subject a second dose of the pharmaceutical composition about one month to about six months after the first dose, and
c. administering to the human subject a third dose of the pharmaceutical composition about three months (e.g., quarterly dosing or dosing every 12 weeks (q12w)) to about six months after the second dose.

In some embodiments, disclosed herein are methods for treating an APOC3-related disease or disorder in a human subject in need thereof, the method comprising:
a. administering to the human subject by subcutaneous administration a first dose of a pharmaceutical composition that comprises the APOC3 RNAi agent at a dose of between about 1 mg to about 100 mg of the APOC3 RNAi Drug Substance,
b. administering to the human subject a second dose of the pharmaceutical composition about three months to about six months after the first dose, and
c. administering to the human subject a third dose of the pharmaceutical composition about six months after the second dose.

In some embodiments, for the methods disclosed herein each dose is between about 10 mg to about 100 mg of the APOC3 RNAi agent.

In some embodiments, for the methods disclosed herein each dose is between about 10 mg to about 50 mg of the APOC3 RNAi agent.

In some embodiments, for the methods disclosed herein each dose is between about 25 mg to about 100 mg of the APOC3 RNAi agent.

In some embodiments, for the methods disclosed herein each dose is between about 25 mg to about 50 mg of the APOC3 RNAi agent.

In some embodiments, for the methods disclosed herein each dose of the APOC3 RNAi agent is about 10 mg.

In some embodiments, for the methods disclosed herein each dose of the APOC3 RNAi agent is about 25 mg.

In some embodiments, for the methods disclosed herein each dose of the APOC3 RNAi agent is about 50 mg.

In some embodiments, for the methods disclosed herein each dose of the APOC3 RNAi agent is about 100 mg.

In some embodiments, for the methods disclosed herein the subject is further administered an additional therapeutic for the treatment of an APOC3-related disease or disorder.

In some embodiments, for the methods disclosed herein the APOC3-related disease or disorder is a dyslipidemia.

In some embodiments, for the methods disclosed herein the APOC3-related disease or disorder is hypertriglyceridemia, obesity, dyslipidemia, non-alcoholic steatohepatitis, non-alcoholic fatty liver disease, hyperlipidemia, abnormal lipid and/or cholesterol metabolism, atherosclerosis, cardiovascular disease, coronary artery disease, hypertriglyceridemia induced pancreatitis, metabolic syndrome, type II diabetes mellitus, familial chylomicronemia syndrome (FCS), chylomicronemia, multifactorial chylomicronemia, or familial partial lipodystrophy.

In some embodiments, for the methods disclosed herein the APOC3-related disease or disorder is familial chylomicronemia syndrome (FCS), chylomicronemia, or multifactorial chylomicronemia.

In some embodiments, for the methods disclosed herein the APOC3-related disease or disorder is hypertriglyceridemia, either with or without a history of pancreatitis.

In some embodiments, for the methods disclosed herein the pharmaceutical composition is packaged in a kit, container, pack, dispenser, pre-filled syringe, or vials.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at an initial dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance described in Table 2 about one month after the initial dose, wherein the initial dose and the second dose are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at an initial dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance described in Table 2 about three months (e.g., quarterly dosing or dosing every 12 weeks (q12w)) after the initial dose, wherein the initial dose and the second dose are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at an initial dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance described in Table 2 about four months after the initial dose, wherein the initial dose and the second dose are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at an initial dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance described in Table 2 about six months after the initial dose, wherein the initial dose and the second dose are administered by subcutaneous injection.

In some embodiments, disclosed herein are methods for treating an APOC3-related disease or disorder in a human subject in need thereof, the method comprising:
a. administering to the human subject by subcutaneous administration an initial dose of a pharmaceutical composition that comprises the APOC3 RNAi Drug Substance described in Table 2 at a dose of between about 1 mg to about 100 mg of the APOC3 RNAi Drug Substance,
b. administering to the human subject a second dose of the pharmaceutical composition about one month after the initial dose, and
c. administering to the human subject a third dose of the pharmaceutical composition about three to about six months after the second dose.

In some embodiments, disclosed herein are methods for treating an APOC3-related disease or disorder in a human subject in need thereof, the method comprising:
a. administering to the human subject by subcutaneous administration an initial dose of a pharmaceutical composition that comprises the APOC3 RNAi Drug Substance described in Table 2 at a dose of between about 1 mg to about 100 mg of the APOC3 RNAi Drug Substance,
b. administering to the human subject a second dose of the pharmaceutical composition about one month after the initial dose, and
c. administering to the human subject a third dose of the pharmaceutical composition about six months after the second dose.

In some embodiments, disclosed herein are methods for treating an APOC3-related disease or disorder in a human subject in need thereof, the method comprising:
a. administering to the human subject by subcutaneous administration an initial dose of a pharmaceutical composition that comprises the APOC3 RNAi Drug Substance described in Table 2 at a dose of between about 1 mg to about 100 mg of the APOC3 RNAi Drug Substance,
b. administering to the human subject a second dose of the pharmaceutical composition about one month to about six months after the initial dose, and
c. administering to the human subject a third dose of the pharmaceutical composition about three months (e.g., quarterly dosing or dosing every 12 weeks (q12w)) to about six months after the second dose.

In some embodiments, disclosed herein are methods for treating an APOC3-related disease or disorder in a human subject in need thereof, the method comprising:
a. administering to the human subject by subcutaneous administration an initial dose of a pharmaceutical composition that comprises the APOC3 RNAi Drug Substance described in Table 2 at a dose of between about 1 mg to about 100 mg of the APOC3 RNAi Drug Substance,
b. administering to the human subject a second dose of the pharmaceutical composition about three months to about six months after the initial dose, and
c. administering to the human subject a third dose of the pharmaceutical composition about six months after the second dose.

In some embodiments, for the methods disclosed herein each dose is between about 10 mg to about 100 mg of the APOC3 RNAi Drug Substance.

In some embodiments, for the methods disclosed herein each dose is between about 10 mg to about 50 mg of the APOC3 RNAi Drug Substance.

In some embodiments, for the methods disclosed herein each dose is between about 25 mg to about 100 mg of the APOC3 RNAi Drug Substance.

In some embodiments, for the methods disclosed herein each dose is between about 25 mg to about 50 mg of the APOC3 RNAi Drug Substance.

In some embodiments, for the methods disclosed herein each dose of the APOC3 RNAi Drug Substance is about 10 mg.

In some embodiments, for the methods disclosed herein each dose of the APOC3 RNAi Drug Substance is about 25 mg.

In some embodiments, for the methods disclosed herein each dose of the APOC3 RNAi Drug Substance is about 50 mg.

In some embodiments, for the methods disclosed herein each dose of the APOC3 RNAi Drug Substance is about 100 mg.

In some embodiments, for the methods disclosed herein the pharmaceutical composition comprises, consists of, or consists essentially of the Formulated APOC3 RNAi Drug Substance described in Table 3.

In some embodiments, for the methods disclosed herein the method further comprises administering additional doses after the first dose, wherein the additional doses are administered about one month apart, about three months apart (such as every 12 weeks apart or once per calendar quarter), about four months apart, or about six months apart.

In some embodiments, for the methods disclosed herein the method further comprises administering additional doses after the second dose, wherein the additional doses are administered about one month apart, about three months apart (such as every 12 weeks apart or once per calendar quarter), about four months apart, or about six months apart.

In some embodiments, for the methods disclosed herein the method further comprises administering additional doses after the third dose, wherein the additional doses are administered about one month apart, about three months apart (such as every 12 weeks apart or once per calendar quarter), about four months apart, or about six months apart.

In some embodiments, for the methods disclosed herein the method further comprises administering additional doses about three to about six months apart.

In some embodiments, disclosed herein are methods of inhibiting expression of APOC3 in a subject in need thereof, the method comprising administering to the subject the APOC3 RNAi Drug Substance described in Table 2, wherein the subject is administered one or more doses in an amount of from about 1 mg to about 100 mg of the APOC3 RNAi Drug Substance described in Table 2 over the course of about one month.

In some embodiments, disclosed herein are methods of inhibiting expression of APOC3 in a subject in need thereof, the method comprising administering to the subject the APOC3 RNAi Drug Substance described in Table 2, wherein the subject is administered one or more doses in an amount of from about 1 mg to about 100 mg of the APOC3 RNAi Drug Substance described in Table 2 over the course of about three months.

In some embodiments, for the methods disclosed herein, the dose is between about 10 mg to about 100 mg of the APOC3 RNAi Drug Substance.

In some embodiments, for the methods disclosed herein, the dose is between about 10 mg to about 50 mg of the APOC3 RNAi Drug Substance.

In some embodiments, for the methods disclosed herein, the dose is between about 25 mg to about 100 mg of the APOC3 RNAi Drug Substance.

In some embodiments, for the methods disclosed herein, the dose is between about 25 mg to about 50 mg of the APOC3 RNAi Drug Substance.

In some embodiments, for the methods disclosed herein, the dose is about 10 mg of the APOC3 RNAi Drug Substance.

In some embodiments, for the methods disclosed herein, the dose is about 25 mg of the APOC3 RNAi Drug Substance.

In some embodiments, for the methods disclosed herein, the dose is about 50 mg of the APOC3 RNAi Drug Substance.

In some embodiments, for the methods disclosed herein, the dose is about 100 mg of the APOC3 RNAi Drug Substance.

In some embodiments, disclosed herein is a pharmaceutical composition for use in treating an APOC3-related disease or disorder in a human subject in need thereof, wherein said use comprises the administration of the pharmaceutical composition comprising the APOC3 RNAi Drug Substance described in Table 2 preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by:

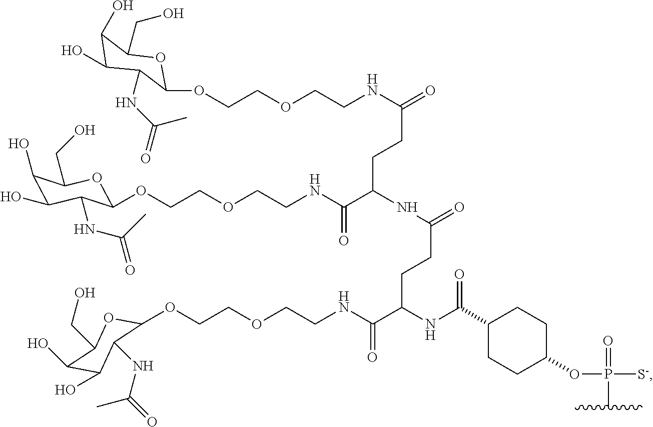

wherein the pharmaceutical composition is administered to the subject at an initial dose of between about 1 mg to about 100 mg of the APOC3 RNAi Drug Substance, and wherein the pharmaceutical composition is administered to the subject at a second dose of between about 1 mg to about 100 mg of the APOC3 RNAi Drug Substance described in Table 2 about one month after the initial dose, wherein the initial dose and the second dose are administered by subcutaneous injection.

In some embodiments, disclosed herein is a pharmaceutical composition for use in treating an APOC3-related disease or disorder in a human subject in need thereof, wherein said use comprises the administration of the pharmaceutical composition comprising the APOC3 RNAi Drug Substance described in Table 2 preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by:

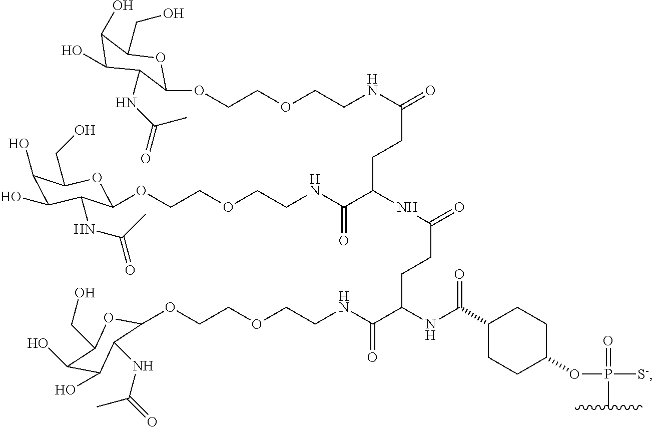

wherein the pharmaceutical composition is administered to the subject at an initial dose of between about 1 mg to about 100 mg of the APOC3 RNAi Drug Substance, and wherein the pharmaceutical composition is administered to the subject at a second dose of between about 1 mg to about 100 mg of the APOC3 RNAi Drug Substance described in Table 2 about three months after the initial dose, wherein the initial dose and the second dose are administered by subcutaneous injection.

In some embodiments, disclosed herein is a pharmaceutical composition for use in treating an APOC3-related disease or disorder in a human subject in need thereof, wherein said use comprises the administration of the pharmaceutical composition comprising the APOC3 RNAi Drug Substance described in Table 2 preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by:

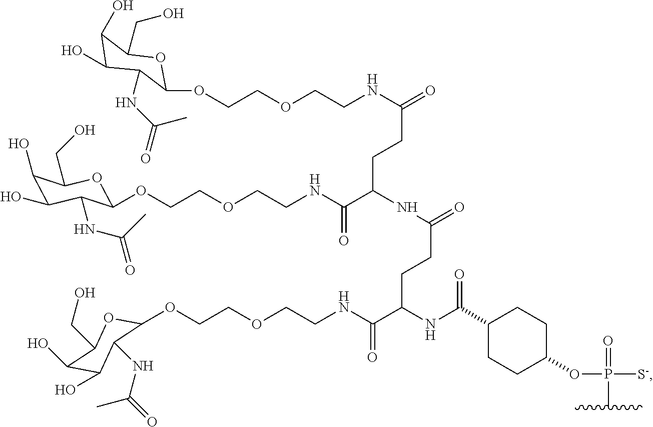

wherein the pharmaceutical composition is administered to the subject at an initial dose of between about 1 mg to about 100 mg of the APOC3 RNAi Drug Substance, and wherein the pharmaceutical composition is administered to the subject at a second dose of between about 1 mg to about 100 mg of the APOC3 RNAi Drug Substance described in Table 2 about four months after the initial dose, wherein the initial dose and the second dose are administered by subcutaneous injection.

In some embodiments, disclosed herein is a pharmaceutical composition for use in treating an APOC3-related disease or disorder in a human subject in need thereof, wherein said use comprises the administration of the pharmaceutical composition comprising the APOC3 RNAi Drug Substance described in Table 2 preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by:

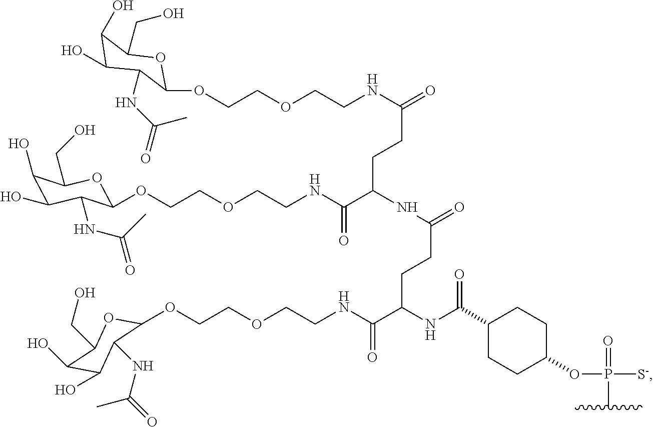

wherein the pharmaceutical composition is administered to the subject at an initial dose of between about 1 mg to about 100 mg of the APOC3 RNAi Drug Substance, and wherein the pharmaceutical composition is administered to the subject at a second dose of between about 1 mg to about 100 mg of the APOC3 RNAi Drug Substance described in Table 2 about six months after the initial dose, wherein the initial dose and the second dose are administered by subcutaneous injection.

In some embodiments, disclosed herein is a pharmaceutical composition for use in treating an APOC3-related disease or disorder in a human subject in need thereof, wherein said use comprises the administration of the pharmaceutical composition that comprises the APOC3 RNAi Drug Substance described in Table 2, wherein:
a. the pharmaceutical composition is administered to the human subject at an initial dose of between about 1 mg to about 100 mg of the APOC3 RNAi Drug Substance,
b. the pharmaceutical composition is administered to the human subject at a second dose of between about 1 mg to about 100 mg of the APOC3 RNAi Drug Substance about one month after the initial dose, and
c. the pharmaceutical composition is administered to the human subject at a third dose of between about 1 mg to about 100 mg of the APOC3 RNAi Drug Substance about three months to about six months after the second dose.

In some embodiments, disclosed herein is a pharmaceutical composition for use in treating an APOC3-related disease or disorder in a human subject in need thereof, wherein said use comprises the administration of the pharmaceutical composition that comprises the APOC3 RNAi Drug Substance described in Table 2, wherein:
a. the pharmaceutical composition is administered to the human subject at an initial dose of between about 1 mg to about 100 mg of the APOC3 RNAi Drug Substance,
b. the pharmaceutical composition is administered to the human subject at a second dose of between about 1 mg to about 100 mg of the APOC3 RNAi Drug Substance about one month after the initial dose, and
c. the pharmaceutical composition is administered to the human subject at a third dose of between about 1 mg to about 100 mg of the APOC3 RNAi Drug Substance about six months after the second dose.

In some embodiments, disclosed herein is a pharmaceutical composition for use in treating an APOC3-related disease or disorder in a human subject in need thereof, wherein said use comprises the administration of the pharmaceutical composition that comprises the APOC3 RNAi Drug Substance described in Table 2, wherein:
a. the pharmaceutical composition is administered to the human subject at an initial dose of between about 1 mg to about 100 mg of the APOC3 RNAi Drug Substance,
b. the pharmaceutical composition is administered to the human subject at a second dose of between about 1 mg to about 100 mg of the APOC3 RNAi Drug Substance about one month to about six months after the initial dose, and
c. the pharmaceutical composition is administered to the human subject at a third dose of between about 1 mg to about 100 mg of the APOC3 RNAi Drug Substance about three months to about six months after the second dose.

A pharmaceutical composition for use in treating an APOC3-related disease or disorder in a human subject in need thereof, wherein said use comprises the administration of the pharmaceutical composition that comprises the APOC3 RNAi Drug Substance described in Table 2, wherein:
a. the pharmaceutical composition is administered to the human subject at an initial dose of between about 1 mg to about 100 mg of the APOC3 RNAi Drug Substance,
b. the pharmaceutical composition is administered to the human subject at a second dose of between about 1 mg to about 100 mg of the APOC3 RNAi Drug Substance about three months to about six months after the initial dose, and
c. the pharmaceutical composition is administered to the human subject at a third dose of between about 1 mg to about 100 mg of the APOC3 RNAi Drug Substance about six months after the second dose.

In some embodiments, for the pharmaceutical compositions when used as disclosed herein each dose of the APOC3 RNAi Drug Substance is between about 10 mg to about 100 mg.

In some embodiments, for the pharmaceutical compositions when used as disclosed herein each dose is between about 10 mg to about 50 mg of the APOC3 RNAi Drug Substance.

In some embodiments, for the pharmaceutical compositions when used as disclosed herein each dose is between about 25 mg to about 100 mg of the APOC3 RNAi Drug Substance.

In some embodiments, for the pharmaceutical compositions when used as disclosed herein each dose is between about 25 mg to about 50 mg of the APOC3 RNAi Drug Substance.

In some embodiments, for the pharmaceutical compositions when used as disclosed herein each dose is about 10 mg of the APOC3 RNAi Drug Substance.

In some embodiments, for the pharmaceutical compositions when used as disclosed herein each dose is about 25 mg of the APOC3 RNAi Drug Substance.

In some embodiments, for the pharmaceutical compositions when used as disclosed herein each dose is about 50 mg of the APOC3 RNAi Drug Substance.

In some embodiments, for the pharmaceutical compositions when used as disclosed herein each dose is about 100 mg of the APOC3 RNAi Drug Substance.

In some embodiments, the pharmaceutical compositions when used as disclosed herein are administered along with an additional therapeutic for the treatment of an APOC3-related disease or disorder to the human subject.

In some embodiments, the pharmaceutical compositions when used as disclosed herein are used to treat a dyslipidemia.

In some embodiments, the pharmaceutical compositions when used as disclosed herein are used to treat hypertriglyceridemia, obesity, dyslipidemia, non-alcoholic steatohepatitis, non-alcoholic fatty liver disease, hyperlipidemia, abnormal lipid and/or cholesterol metabolism, atherosclerosis, cardiovascular disease, coronary artery disease, hypertriglyceridemia induced pancreatitis, metabolic syndrome, type II diabetes mellitus, familial chylomicronemia syndrome (FCS), chylomicronemia, multifactorial chylomicronemia, or familial partial lipodystrophy.

In some embodiments, the pharmaceutical compositions when used as disclosed herein are used to treat familial chylomicronemia syndrome (FCS), chylomicronemia, or multifactorial chylomicronemia.

In some embodiments, the pharmaceutical compositions when used as disclosed herein are used to treat hypertriglyceridemia, either with or without a history of pancreatitis.

In some embodiments, the pharmaceutical compositions when used as disclosed herein are packaged in a kit, container, pack, dispenser, pre-filled syringe, or vials.

In some embodiments, the pharmaceutical compositions when used as disclosed herein comprise, consist of, or consist essentially of the Formulated APOC3 RNAi Drug Substance described in Table 3.

In some embodiments, the pharmaceutical compositions when used as disclosed herein are administered to the subject in additional doses that are administered about one month apart.

In some embodiments, the pharmaceutical compositions when used as disclosed herein are administered to the subject in additional doses that are administered about three months apart.

In some embodiments, the pharmaceutical compositions when used as disclosed herein are administered to the subject in additional doses that are administered about four months apart.

In some embodiments, the pharmaceutical compositions when used as disclosed herein are administered to the subject in additional doses that are administered about six months apart.

In some embodiments, the pharmaceutical compositions when used as disclosed herein are administered to the subject in additional doses that are administered about three to about six months apart.

In some embodiments, the pharmaceutical compositions when used as disclosed herein are self-administered by the subject.

In some embodiments, the pharmaceutical compositions when used as disclosed herein are self-administered by a medical professional.

In some embodiments, the pharmaceutical compositions when used as disclosed herein reduce the serum APOC3 protein levels in the human subject by greater than 60% compared to baseline levels.

In some embodiments, the pharmaceutical compositions when used as disclosed herein reduce the serum triglyceride (TG) levels in the human subject by greater than 50% compared to baseline levels.

In some embodiments, the pharmaceutical compositions when used as disclosed herein reduce the serum triglyceride (TG) levels by greater than 75% in the human subject.

In some embodiments, the pharmaceutical compositions when used as disclosed herein reduce very low density lipoprotein cholesterol (VLDL-C) levels, the serum low density lipoprotein cholesterol (LDL-C) levels, or both the serum VLDL-C and LDL-C levels in the human subject compared to baseline levels.

In some embodiments, the pharmaceutical compositions when used as disclosed herein are comprised of an APOC3 RNAi Drug Substance that is in the form of a salt, a mixed salt, a free acid, or a combination thereof.

In some embodiments, the pharmaceutical compositions when used as disclosed herein are comprised of an APOC3 RNAi Drug Substance that is in the form of a pharmaceutically acceptable sodium salt.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 1 mg to about 100 mg of the APOC3 RNAi Drug Substance described in Table 2 over the course of about one month.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 1 mg to about 100 mg of the APOC3 RNAi Drug Substance described in Table 2 over the course of about three months.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 10 mg to about 100 mg of the APOC3 RNAi Drug Substance described in Table 2 over the course of about one month.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 10 mg to about 100 mg of the APOC3 RNAi Drug Substance described in Table 2 over the course of about three months.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 10 mg to about 100 mg of the APOC3 RNAi Drug Substance described in Table 2 over the course of about six months.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 25 mg to about 50 mg of the APOC3 RNAi Drug Substance described in Table 2 over the course of about one month.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 25 mg to about 50 mg of the APOC3 RNAi Drug Substance described in Table 2 over the course of about three months.

In some embodiments, disclosed herein are pharmaceutical compositions for inhibiting expression of APOC3 in a human subject in need thereof by administration of the pharmaceutical composition comprising the APOC3 RNAi Drug Substance described in Table 2, wherein the pharmaceutical composition is administered in one or more doses in an amount of from about 25 mg to about 50 mg of the APOC3 RNAi Drug Substance described in Table 2 over the course of about six months.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 100 mg of the APOC3 RNAi Drug Substance, and administering to the subject subsequent doses, wherein at least one month passes between doses, wherein the doses are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 100 mg of the APOC3 RNAi Drug Substance, and administering to the subject subsequent doses, wherein at least about twelve weeks passes between doses (q12w dosing), wherein the doses are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 100 mg of the APOC3 RNAi Drug Substance, and administering to the subject subsequent doses, wherein at least about three months passes between doses, wherein the doses are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 100 mg of the APOC3 RNAi Drug Substance, and administering to the subject subsequent doses, wherein at least about six months passes between doses, wherein the doses are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 50 mg of the APOC3 RNAi Drug Substance, and administering to the subject subsequent doses, wherein at least one month passes between doses, wherein the doses are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 50 mg of the APOC3 RNAi Drug Substance, and administering to the subject subsequent doses, wherein at least about twelve weeks passes between doses (q12w dosing), wherein the doses are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 50 mg of the APOC3 RNAi Drug Substance, and administering to the subject subsequent doses, wherein at least about three months passes between doses, wherein the doses are administered by subcutaneous injection.

In some embodiments, disclosed herein are methods for treating an APOC3-related disease or disorder in a human subject in need thereof, the method comprising administering to the subject a pharmaceutical composition that comprises the APOC3 RNAi Drug Substance described in Table 2 at a dose of no greater than about 50 mg of the APOC3 RNAi Drug Substance, and administering to the subject subsequent doses, wherein at least about six months passes between doses, wherein the doses are administered by subcutaneous injection.

In some embodiments, disclosed herein are methods for treating an APOC3-related disease or disorder in a human subject in need thereof, the method comprising:
a. administering to the human subject a dose of a pharmaceutical composition that comprises no greater than about 100 mg of the APOC3 RNAi Drug Substance,
b. administering to the human subject a second dose of the pharmaceutical composition about one month to about six months after the initial dose, and
c. administering to the human subject a third dose of the pharmaceutical composition about six months after the second dose, wherein the doses are administered by subcutaneous administration.

In some embodiments, disclosed herein are methods for treating an APOC3-related disease or disorder in a human subject in need thereof, the method comprising:
a. administering to the human subject a dose of a pharmaceutical composition that comprises no greater than about 100 mg of the APOC3 RNAi Drug Substance,
b. administering to the human subject a second dose of the pharmaceutical composition about one month after the initial dose, and
c. administering to the human subject a third dose of the pharmaceutical composition about three months (e.g., dosing every calendar quarter or once every 12 weeks (q12w)) to about six months after the second dose,
   wherein the doses are administered by subcutaneous administration.

In some embodiments, disclosed herein are methods for treating an APOC3-related disease or disorder in a human subject in need thereof, the method comprising:
a. administering to the human subject a dose of a pharmaceutical composition that comprises no greater than about 100 mg of the APOC3 RNAi Drug Substance,
b. administering to the human subject a second dose of the pharmaceutical composition about three months (e.g., dosing every calendar quarter or once every 12 weeks (q12w)) to about six months after the initial dose, and
c. administering to the human subject a third dose of the pharmaceutical composition about six months after the second dose,
   wherein the doses are administered by subcutaneous administration.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating an APOC3-related disease or disorder in a human subject in need thereof, the method comprising administering to the subject a pharmaceutical composition that comprises APOC3 RNAi Drug Substance preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by: 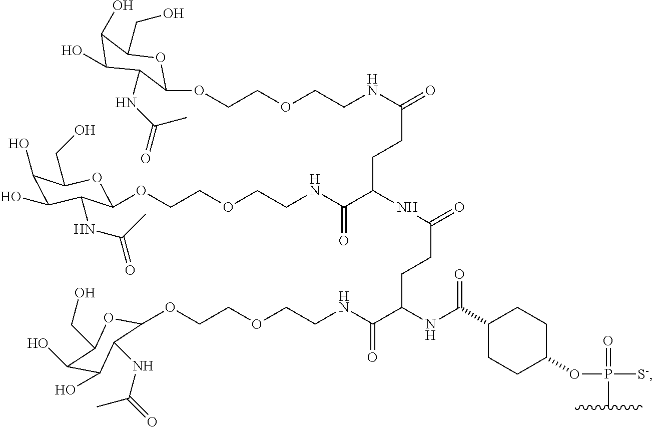
wherein the APOC3 RNAi Drug Substance is administered a first dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance about one month after the first dose, wherein the first dose and the second dose are administered by subcutaneous injection.

2. A method of treating an APOC3-related disease or disorder in a human subject in need thereof, the method comprising administering to the subject a pharmaceutical composition that comprises APOC3 RNAi Drug Substance preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by: 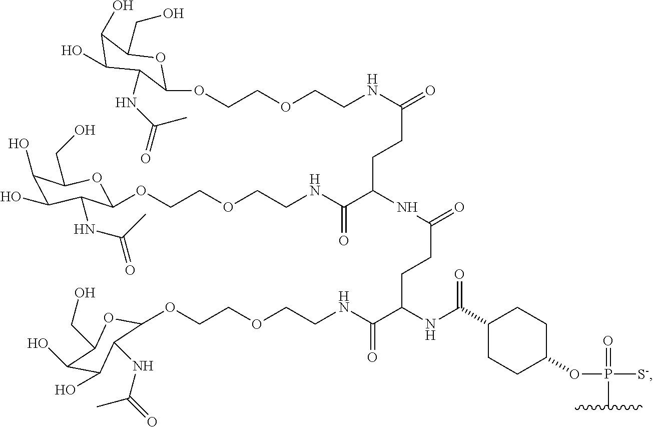
wherein the APOC3 RNAi Drug Substance is administered a first dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance about three months after the first dose, wherein the first dose and the second dose are administered by subcutaneous injection.

3. A method of treating an APOC3-related disease or disorder in a human subject in need thereof, the method comprising administering to the subject a pharmaceutical composition that comprises APOC3 RNAi Drug Substance preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by: 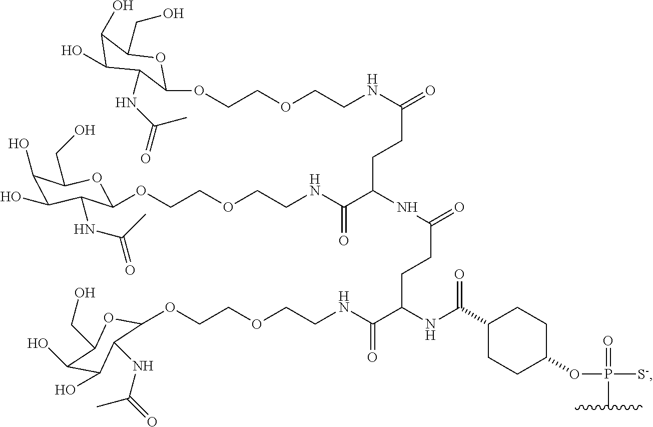
wherein the APOC3 RNAi Drug Substance is administered a first dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance about four months after the first dose, wherein the first dose and the second dose are administered by subcutaneous injection.

4. A method of treating an APOC3-related disease or disorder in a human subject in need thereof, the method comprising administering to the subject a pharmaceutical composition that comprises APOC3 RNAi Drug Substance preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by: 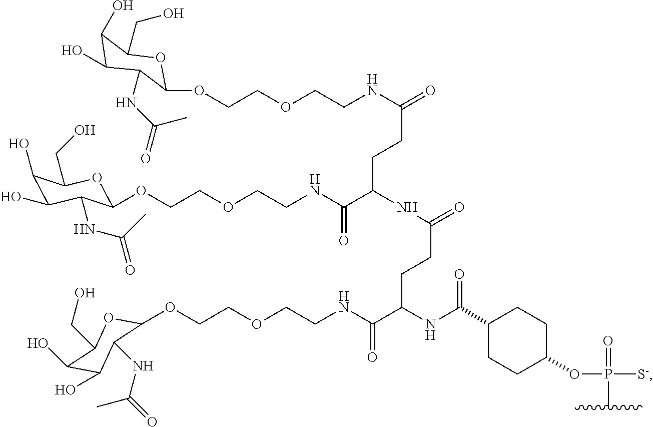
wherein the APOC3 RNAi Drug Substance is administered a first dose of between about 1 mg to about 100 mg of the APOC3 RNAi Drug Substance, and administering to the subject a second dose of the pharmaceutical composition comprising between about 1 mg to about 100 mg of the APOC3 RNAi Drug Substance, about six months after the first dose, wherein the first dose and the second dose are administered by subcutaneous injection.

5. The method of any one of claims 1-4, further comprising administering additional doses after the second dose, wherein the additional doses are administered about one month apart.

6. The method of any one of claims 1-4, further comprising administering additional doses after the second dose, wherein the additional doses are administered about three months apart.

7. The method of any one of claims 1-4, further comprising administering additional doses after the second dose, wherein the additional doses are administered about four months apart.

8. The method of any one of claims 1-4, further comprising administering additional doses after the second dose, wherein the additional doses are administered about six months apart.

9. A method of treating an APOC3-related disease or disorder in a human subject in need thereof, the method comprising:
a. administering to the human subject a first dose of a pharmaceutical composition that comprises APOC3 RNAi Drug Substance preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by: 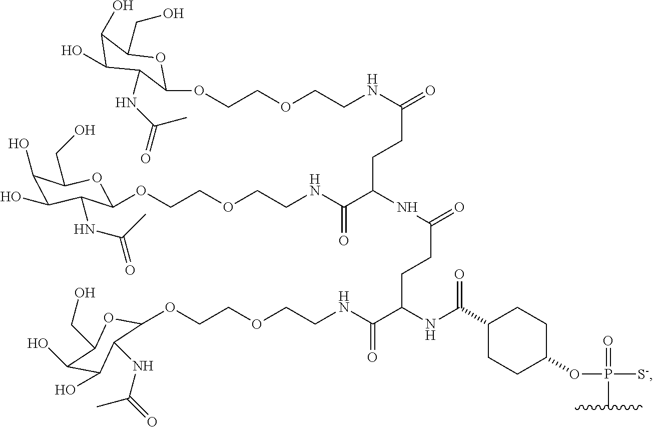
wherein the APOC3 RNAi Drug Substance is administered at a dose of between about 1 mg to about 100 mg of the APOC3 RNAi Drug Substance,
b. administering to the human subject a second dose of the pharmaceutical composition about one month to about six months after the first dose, and
c. administering to the human subject a third dose of the pharmaceutical composition about three months to about six months after the second dose,
wherein the first dose, the second dose, and the third dose are each administered by subcutaneous injection.

10. The method of claim 5, wherein the second dose is administered about one month after the first dose.

11. The method of claim 5, wherein the second dose is administered about three months after the first dose.

12. The method of claim 5, wherein the second dose is administered about four months after the first dose.

13. The method of claim 5, wherein the second dose is administered about six months after the first dose.

14. The method of any one of claims 5-13, wherein the third dose is administered about three months after the second dose.

15. The method of any one of claims 5-13, wherein the third dose is administered about four months after the second dose.

16. The method of any one of claims 5-13, wherein the third dose is administered about six months after the second dose.

17. The method of any one of claims 5-13, further comprising administering additional doses after the third dose, wherein the additional doses are administered about one month to about six months apart.

18. The method of any one of claims 5-13, further comprising administering additional doses after the third dose, wherein the additional doses are administered about three months to about six months apart.

19. The method of any one of claims 5-13, further comprising administering additional doses after the third dose, wherein the additional doses are administered about six months apart.

20. The method of any one of claims 1-19, wherein each dose is between about 10 mg to about 100 mg of the APOC3 RNAi Drug Substance.

21. The method of any one of claims 1-19, wherein each dose is between about 10 mg to about 50 mg of the APOC3 RNAi Drug Substance.

22. The method of any one of claims 1-19, wherein each dose is between about 25 mg to about 100 mg of the APOC3 RNAi Drug Substance.

23. The method of any one of claims 1-19, wherein each dose is between about 25 mg to about 50 mg of the APOC3 RNAi Drug Substance.

24. The method of any one of claims 1-19, wherein each dose of the APOC3 RNAi Drug Substance is about 10 mg.

25. The method of any one of claims 1-19, wherein each dose of the APOC3 RNAi Drug Substance is about 25 mg.

26. The method of any one of claims 1-19, wherein each dose of the APOC3 RNAi Drug Substance is about 50 mg.

27. The method of any one of claims 1-19, wherein each dose of the APOC3 RNAi Drug Substance is about 100 mg.

28. The method of any one of claims 1-27, wherein the subject is further administered an additional therapeutic for the treatment of an APOC3-related disease or disorder.

29. The method of any one of claims 1-28, wherein the APOC3-related disease or disorder is a dyslipidemia.

30. The method of any one of claims 1-28, wherein the APOC3-related disease or disorder is hypertriglyceridemia, obesity, dyslipidemia, non-alcoholic steatohepatitis, non-alcoholic fatty liver disease, hyperlipidemia, abnormal lipid and/or cholesterol metabolism, atherosclerosis, cardiovascular disease, coronary artery disease, hypertriglyceridemia induced pancreatitis, metabolic syndrome, type II diabetes mellitus, familial chylomicronemia syndrome (FCS), chylomicronemia, multifactorial chylomicronemia, lipodystrophy syndromes, or familial partial lipodystrophy.

31. The method of any one of claims 1-28, wherein the APOC3-related disease or disorder is chylomicronemia.

32. The method of any one of claims 1-28, wherein the APOC3-related disease or disorder is hypertriglyceridemia, either with or without a history of pancreatitis.

33. The method of any one of claims 1-32, wherein the pharmaceutical composition is packaged in a kit, container, pack, dispenser, pre-filled syringe, or vials.

34. The method of any one of claims 1-33, wherein the pharmaceutical composition comprises, consists of, or consists essentially of the Formulated APOC3 RNAi Drug Substance described in Table 3 comprised of the APOC3 RNAi Drug Substance in a concentration of about 200 mg/mL, sodium phosphate monobasic monohydrate in a concentration of about 0.062 mg/mL, anhydrous sodium phosphate dibasic in a concentration of about 0.063 mg/mL, and water in a concentration of about 890 mg/mL.

35. The method of any one of claims 1-34, wherein the administration of one or more doses of the pharmaceutical composition is performed by the subject.

36. The method of any one of claims 1-35, wherein the administration of one or more doses of the pharmaceutical composition is performed by a medical professional.

37. The method of any one of claims 1-36, wherein the serum APOC3 protein levels are reduced by greater than 60% from baseline levels in the human subject.

38. The method of any one of claims 1-37, wherein the serum triglyceride (TG) levels are reduced by greater than 50% from baseline levels in the human subject.

39. The method of claim 38, wherein the serum TG levels are reduced by greater than 75% from baseline levels in the human subject.

40. The method of any one of claims 1-39, wherein the serum very low density lipoprotein cholesterol (VLDL-C) levels, the serum low density lipoprotein cholesterol (LDL-C) levels, or both the serum VLDL-C and LDL-C levels are reduced in the human subject compared to baseline levels.

41. The method of any one of claims 1-40, wherein the subject has fasting or post-prandial baseline triglyceride levels greater than 150 mg/dL.

42. The method of any one of claims 1-41, wherein the subject has fasting or post-prandial baseline triglyceride levels greater than 500 mg/dL.

43. The method of any one of claims 1-42, wherein the subject has fasting or post-prandial baseline triglyceride levels greater than 1000 mg/dL.

44. The method of any one of claim 1-33 or 35-43, wherein the APOC3 RNAi Drug Substance is administered as a pharmaceutically acceptable salt, pharmaceutically acceptable mixed salt, free acid, or a combination thereof.

45. The method of claim 44, wherein the APOC3 RNAi Drug Substance is administered as a pharmaceutically acceptable sodium salt.

46. A unit dosage form comprising APOC3 RNAi Drug Substance preferably in a pharmaceutically acceptable salt form, the APOC3 RNAi Drug Substance comprising an antisense strand comprising the nucleotide sequence: usCfsasCfuGfagaauAfcUfgUfcCfcGfsu (SEQ ID NO:2), and a sense strand comprising the nucleotide sequence: (NAG37)s(invAb)sacgggacaGfUfAfuucucaguias(invAb) (SEQ ID NO:6), wherein a, c, g, i, and u represent 2′-O-methyl adenosine, cytidine, guanosine, inosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue; and (NAG37)s comprises the structure represented by: 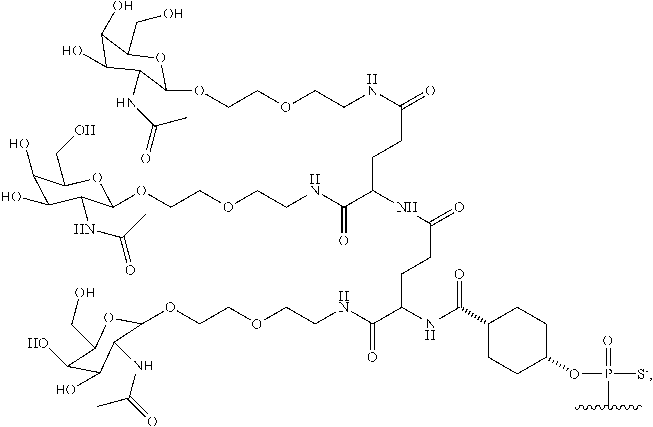
at an amount of between about 1 mg to about 100 mg of the APOC3 RNAi Drug Substance.

47. The unit dosage form of claim 46, wherein the amount of APOC3 RNAi Drug Substance is from about 10 mg to about 50 mg.

48. The unit dosage form of claim 46, wherein the amount of APOC3 RNAi Drug Substance is from about 10 mg to about 25 mg.

49. A method of inhibiting expression of APOC3 in a subject in need thereof, the method comprising administering to the subject the APOC3 RNAi Drug Substance described in Table 2, wherein the subject is administered one or more doses in an amount of from about 1 mg to about 100 mg of the APOC3 RNAi Drug Substance described in Table 2:
a. over the course of about one month; or
b. over the course of about three months, or
c. over the course of about six months.

50. The method of claim 49, wherein the dose is between about 10 mg to about 100 mg of the APOC3 RNAi Drug Substance.

51. The method of claim 49, wherein the dose is between about 10 mg to about 50 mg of the APOC3 RNAi Drug Substance.

52. The method of claim 49, wherein the dose is between about 25 mg to about 100 mg of the APOC3 RNAi Drug Substance.

53. The method of claim 49, wherein the dose is between about 25 mg to about 50 mg of the APOC3 RNAi Drug Substance.

54. The method of claim 49, wherein the dose is about 10 mg of the APOC3 RNAi Drug Substance.

55. The method of claim 49, wherein the dose is about 25 mg of the APOC3 RNAi Drug Substance.

56. The method of claim 49, wherein the dose is about 50 mg of the APOC3 RNAi Drug Substance.

57. The method of claim 49, wherein the dose is about 100 mg of the APOC3 RNAi Drug Substance.

58. A pharmaceutical composition for use in treating an APOC3-related disease or disorder in a human subject in need thereof, wherein said use comprises the administration of the pharmaceutical composition comprising an APOC3 RNAi agent, wherein the pharmaceutical composition is administered to the subject at a first dose of between about 1 mg to about 100 mg of the APOC3 RNAi agent, and wherein the pharmaceutical composition is administered to the subject at a second dose of between about 1 mg to about 100 mg of the APOC3 RNAi agent about one month after the first dose, wherein the first dose and the second dose are administered by subcutaneous injection.

59. A pharmaceutical composition for use in treating an APOC3-related disease or disorder in a human subject in need thereof, wherein said use comprises the administration of the pharmaceutical composition comprising an APOC3 RNAi agent, wherein the pharmaceutical composition is administered to the subject at a first dose of between about 1 mg to about 100 mg of the APOC3 RNAi agent, and wherein the pharmaceutical composition is administered to the subject at a second dose of between about 1 mg to about 100 mg of the APOC3 RNAi agent about three months after the first dose, wherein the first dose and the second dose are administered by subcutaneous injection.

60. A pharmaceutical composition for use in treating an APOC3-related disease or disorder in a human subject in need thereof, wherein said use comprises the administration of the pharmaceutical composition comprising an APOC3 RNAi agent, wherein the pharmaceutical composition is administered to the subject at a first dose of between about 1 mg to about 100 mg of the APOC3 RNAi agent, and wherein the pharmaceutical composition is administered to the subject at a second dose of between about 1 mg to about 100 mg of the APOC3 RNAi agent, about four months after the first dose, wherein the first dose and the second dose are administered by subcutaneous injection.

61. A pharmaceutical composition for use in treating an APOC3-related disease or disorder in a human subject in need thereof, wherein said use comprises the administration of the pharmaceutical composition comprising an APOC3 RNAi agent, wherein the pharmaceutical composition is administered to the subject at a first dose of between about 1 mg to about 100 mg of the APOC3 RNAi agent, and wherein the pharmaceutical composition is administered to the subject at a second dose of between about 1 mg to about 100 mg of the APOC3 RNAi agent about six months after the first dose, wherein the first dose and the second dose are administered by subcutaneous injection.

62. A pharmaceutical composition for use in treating an APOC3-related disease or disorder in a human subject in need thereof, wherein said use comprises the administration of the pharmaceutical composition comprising an APOC3 RNAi agent, wherein:
a. the pharmaceutical composition is administered to the human subject at a first dose of between about 1 mg to about 100 mg of the APOC3 RNAi agent,
b. the pharmaceutical composition is administered to the human subject at a second dose of between about 1 mg to about 100 mg of the APOC3 RNAi agent about one month after the first dose, and
c. the pharmaceutical composition is administered to the human subject at a third dose of between about 1 mg to about 100 mg of the APOC3 RNAi agent about three months to about six months after the second dose.

63. A pharmaceutical composition for use in treating an APOC3-related disease or disorder in a human subject in need thereof, wherein said use comprises the administration of the pharmaceutical composition comprising an APOC3 RNAi agent, wherein:
a. the pharmaceutical composition is administered to the human subject at a first dose of between about 1 mg to about 100 mg of the APOC3 RNAi agent,
b. the pharmaceutical composition is administered to the human subject at a second dose of between about 1 mg to about 100 mg of the APOC3 RNAi agent about one month after the first dose, and
c. the pharmaceutical composition is administered to the human subject at a third dose of between about 1 mg to about 100 mg of the APOC3 RNAi agent about six months after the second dose.

64. A pharmaceutical composition for use in treating an APOC3-related disease or disorder in a human subject in need thereof, wherein said use comprises the administration of the pharmaceutical composition comprising an APOC3 RNAi agent, wherein:
a. the pharmaceutical composition is administered to the human subject at a first dose of between about 1 mg to about 100 mg of the APOC3 RNAi agent,
b. the pharmaceutical composition is administered to the human subject at a second dose of between about 1 mg to about 100 mg of the APOC3 RNAi agent about one month to about six months after the first dose, and
c. the pharmaceutical composition is administered to the human subject at a third dose of between about 1 mg to about 100 mg of the APOC3 RNAi agent about three months to about six months after the second dose.

65. A pharmaceutical composition for use in treating an APOC3-related disease or disorder in a human subject in need thereof, wherein said use comprises the administration of the pharmaceutical composition comprising an APOC3 RNAi agent, wherein:
a. the pharmaceutical composition is administered to the human subject at a first dose of between about 1 mg to about 100 mg of the APOC3 RNAi agent,
b. the pharmaceutical composition is administered to the human subject at a second dose of between about 1 mg to about 100 mg of the APOC3 RNAi agent about three months to about six months after the first dose, and
c. the pharmaceutical composition is administered to the human subject at a third dose of between about 1 mg to about 100 mg of the APOC3 RNAi agent about six months after the second dose.

66. The pharmaceutical composition of any one of claims 58-65, wherein each dose of the APOC3 RNAi agent is between about 10 mg to about 100 mg.

67. The pharmaceutical composition of any one of claims 58-65, wherein each dose is between about 10 mg to about 50 mg of the APOC3 RNAi agent.

68. The pharmaceutical composition of any one of claims 58-65, wherein each dose is between about 25 mg to about 100 mg of the APOC3 RNAi agent.

69. The pharmaceutical composition of any one of claims 58-65, wherein each dose is between about 25 mg to about 50 mg of the APOC3 RNAi agent.

70. The pharmaceutical composition of any one of claims 58-65, wherein each dose is about 10 mg of the APOC3 RNAi agent.

71. The pharmaceutical composition of any one of claims 58-65, wherein each dose is about 25 mg of the APOC3 RNAi agent.

72. The pharmaceutical composition of any one of claims 58-65, wherein each dose is about 50 mg of the APOC3 RNAi agent.

73. The pharmaceutical composition of any one of claims 58-65, wherein each dose is about 100 mg of the APOC3 RNAi agent.

74. The pharmaceutical composition of any one of claims 58-73, wherein an additional therapeutic for the treatment of an APOC3-related disease or disorder is administered to the human subject.

75. The pharmaceutical composition of any one of claims 58-74, wherein the APOC3-related disease or disorder is a dyslipidemia.

76. The pharmaceutical composition of any one of claims 58-74, wherein the APOC3-related disease or disorder is hypertriglyceridemia, obesity, dyslipidemia, non-alcoholic steatohepatitis, non-alcoholic fatty liver disease, hyperlipidemia, abnormal lipid and/or cholesterol metabolism, atherosclerosis, cardiovascular disease, coronary artery disease, hypertriglyceridemia induced pancreatitis, metabolic syndrome, type II diabetes mellitus, familial chylomicronemia syndrome (FCS), chylomicronemia, multifactorial chylomicronemia, lipodystrophy syndromes, or familial partial lipodystrophy.

77. The pharmaceutical composition of any one of claims 58-74, wherein the APOC3-related disease or disorder is familial chylomicronemia syndrome, chylomicronemia, or multifactorial chylomicronemia.

78. The pharmaceutical composition of any one of claims 58-74, wherein the APOC3-related disease or disorder is hypertriglyceridemia, either with or without a history of pancreatitis.

79. The pharmaceutical composition of any one of claims 58-78, wherein the pharmaceutical composition is packaged in a kit, container, pack, dispenser, pre-filled syringe, or vials.

80. The pharmaceutical composition of any one of claims 58-79, wherein the pharmaceutical composition comprises, consists of, or consists essentially of the Formulated APOC3 RNAi Drug Substance described in Table 3 comprised of the APOC3 RNAi Drug Substance in a concentration of about 200 mg/mL, sodium phosphate monobasic monohydrate in a concentration of about 0.062 mg/mL, anhydrous sodium phosphate dibasic in a concentration of about 0.063 mg/mL, and water in a concentration of about 890 mg/mL.

81. The pharmaceutical composition of any one of claims 48-80, wherein the pharmaceutical composition is further administered to the subject in additional doses that are administered about one month apart.

82. The pharmaceutical composition of any one of claims 48-80, wherein the pharmaceutical composition is further administered to the subject in additional doses that are administered about three months apart.

83. The pharmaceutical composition of any one of claims 48-80, wherein the pharmaceutical composition is further administered to the subject in additional doses that are administered about four months apart.

84. The pharmaceutical composition of any one of claims 48-80, wherein the pharmaceutical composition is further administered to the subject in additional doses after the second dose, wherein the additional doses are administered about six months apart.

85. The pharmaceutical composition of any one of claims 48-80, wherein the pharmaceutical composition is further administered to the subject in additional doses after the third dose, wherein the additional doses are administered about three to about six months apart.

86. The pharmaceutical composition of any one of claims 48-85, wherein the administration of one or more doses of the pharmaceutical composition is performed by the subject.

87. The pharmaceutical composition of any one of claims 48-85, wherein the administration of one or more doses of the pharmaceutical composition is performed by a medical professional.

88. The pharmaceutical composition of any one of claims 48-87, wherein the serum APOC3 protein levels are reduced by greater than 60% in the human subject compared to baseline levels.

89. The pharmaceutical composition of any one of claims 48-88, wherein the serum triglyceride (TG) levels are reduced by greater than 50% in the human subject compared to baseline levels.

90. The pharmaceutical composition of claim 89, wherein the serum TG levels are reduced by greater than 75% in the human subject compared to baseline levels.

91. The pharmaceutical composition of any one of claims 48-90, wherein the serum very low density lipoprotein cholesterol (VLDL-C) levels, the serum low density lipoprotein cholesterol (LDL-C) levels, or both the serum VLDL-C and LDL-C levels are reduced in the human subject compared to baseline levels.

92. The pharmaceutical composition of any one of claim 48-79 or 81-91, wherein the APOC3 RNAi agent is administered as a salt, a mixed salt, a free acid, or a combination thereof.

93. The pharmaceutical composition of claim 92, wherein the APOC3 RNAi agent is administered as a pharmaceutically acceptable sodium salt.

94. A pharmaceutical composition for inhibiting expression of APOC3 in a human subject in need thereof by administration of the pharmaceutical composition comprising an APOC3 RNAi agent, wherein the pharmaceutical composition is administered in one or more doses in an amount of from about 1 mg to about 100 mg of the APOC3 RNAi agent:
a. over the course of about one month, or
b. over the course of about three months, or
c. over the course of about six months.

95. The pharmaceutical composition of claim 94, wherein the dose is between about 10 mg to about 100 mg of the APOC3 RNAi agent.

96. The pharmaceutical composition of claim 94, wherein the dose is between about 10 mg to about 50 mg of the APOC3 RNAi agent.

97. The pharmaceutical composition of claim 94, wherein the dose is between about 25 mg to about 100 mg of the APOC3 RNAi agent.

98. The pharmaceutical composition of claim 94, wherein the dose is between about 25 mg to about 50 mg of the APOC3 RNAi agent.

99. The pharmaceutical composition of claim 94, wherein the dose is about 10 mg of the APOC3 RNAi agent.

100. The pharmaceutical composition of claim 94, wherein the dose is about 25 mg of the APOC3 RNAi agent.

101. The pharmaceutical composition of claim 94, wherein the dose is about 50 mg of the APOC3 RNAi agent.

102. The pharmaceutical composition of claim 94, wherein the dose is about 100 mg of the APOC3 RNAi agent.


SEQUENCE LISTING (6 sequences)

SEQ ID NO: 1 - DNA, 533 bp, Homo sapiens
  Homo Sapiens apolipoprotein C3 (APOC3) transcript, GenBank NM_000040.1
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 2 - RNA, 21 nt, Artificial sequence
  APOC3 RNAi agent antisense strand modified sequence
ucacugagaauacugucccgu

SEQ ID NO: 3 - RNA, 21 nt, Artificial sequence
  APOC3 RNAi agent antisense strand underlying base sequence
ucacugagaauacugucccgu

SEQ ID NO: 4 - RNA, 21 nt, Artificial sequence
  APOC3 RNAi agent sense strand modified sequence
  modified_base 20: n = 2′-O-methylinosine-3′-phosphate
acgggacaguauucucaguna

SEQ ID NO: 5 - RNA, 21 nt, Artificial sequence
  APOC3 RNAi agent sense strand underlying base sequence
  modified_base 20: n = inosine (hypoxanthine)
acgggacaguauucucaguna

SEQ ID NO: 6 - RNA, 21 nt, Artificial sequence
  APOC3 RNAi agent sense strand modified sequence
  modified_base 20: n = 2′-O-methylinosine-3′-phosphate
acgggacaguauucucaguna